(19) United States
(12) Patent Application Publication
(10) Pub. No.: US 2011/0305713 A1
(43) Pub. Date: Dec. 15, 2011

(54) METHODS AND COMPOSITIONS TO ENHANCE VACCINE EFFICACY BY REPROGRAMMING REGULATORY T CELLS

(75) Inventors: David H. MUNN, Augusta, GA (US); Andrew L. Mellor, Augusta, GA (US); Madhav D. Sharma, Augusta, GA (US); Yukai He, Evans, GA (US)

(73) Assignee: Medical College of Georgia Research Institute, Inc, Augusta, GA (US)

(21) Appl. No.: 13/086,090

(22) Filed: Apr. 13, 2011

Related U.S. Application Data
(63) Continuation-in-part of application No. 12/083,855, filed on Jul. 20, 2009.
Application No. 12/083,855 is a continuation-in-part of application No. 12/158,170, filed on Oct. 20, 2008.
(60) Provisional application No. 60/729,041, filed on Oct. 21, 2005.
(60) Provisional application No. 60/756,861, filed on Jan. 7, 2006.
(60) Provisional application No. 61/323,641, filed on Apr. 13, 2010.

(51) Int. Cl. A61K 39/395; A61K 31/7088; A61P 37/02; C12N 5/0783; A61K 39/21; A61K 39/00

(52) U.S. Cl. 424/173.1; 424/199.1; 424/184.1; 435/375; 514/44 R

(57) ABSTRACT

The immunoregulatory enzyme indoleamine 2,3-dioxygenase (IDO) is expressed by a subset of murine plasmacytoid DCs (pDCs) in tumor-draining LNs, where it can potently activate Foxp3 regulatory T cells (Tregs). We now show that IDO functions as a molecular switch in tumor-draining LNs, maintaining Tregs in their normal suppressive phenotype when IDO was active, but allowing inflammation-induced conversion of Tregs to a polyfunctional T-helper phenotype similar to proinflammatory TH17 cells when IDO was blocked. In vitro, conversion of Tregs to the TH17-like phenotype was driven by antigen-activated effector T cells, and required IL-6 produced by activated pDCs. IDO regulated this conversion by dominantly suppressing production of IL-6 in pDCs, in a GCN2-kinase dependent fashion. In vivo, using a model of established B16 melanoma, the combination of an IDO-inhibitor drug plus anti-tumor vaccine caused upregulation of IL-6 in pDCs and in situ conversion of a majority of Tregs to the TH17 phenotype, with marked enhancement of CD8⁺ T cell activation and anti-tumor efficacy. Thus, Tregs in tumor-draining LNs can be actively re-programmed in vitro and in vivo into T-helper cells, without the need for physical depletion, and IDO serves as a key regulator of this critical conversion.

This application is a continuation-in-part of U.S. application Ser. No. 12/083,855 filed Jul. 20, 2009 which is §371 U.S. National Stage of International Application No. PCT/US2006/040796, filed Oct. 20, 2006, which claims priority to U.S. Provisional Application No. 60/729,041, filed Oct. 21, 2005. This application is also a continuation-in-part of U.S. application Ser. No. 12/158,170 filed Oct. 20, 2008 which is §371 U.S. National Stage of International Application No.PCT/US07/00404 filed Jan. 5, 2007, which claims priority to U.S. Provisional Application No. 60/756,861 filed Jan. 7, 2006. This application also claims priority to U.S. Provisional Application No. 61/323,641, filed on Apr. 13, 2010, the contents of each which are herein incorporated by reference in their entireties.

### BACKGROUND

A recently discovered molecular mechanism contributing to peripheral immune tolerance is the immunoregulatory enzyme indoleamine 2,3-dioxygenase (IDO). Cells expressing the tryptophan-catabolizing enzyme IDO are capable of inhibiting T cell proliferation in vitro and reducing T cell immune responses in vivo (See e.g U.S. Pat. Nos. 6,451,840 and 6,482,416).

The IDO enzyme is well characterized and compounds that serve as substrates or inhibitors of the IDO enzyme are known. For example, Southan (Southan et al, Med. Chem. Res., 1996; 343-352) utilized an in vitro assay system to identify tryptophan analogues that serve as either substrates or inhibitors of human IDO. Methods for detecting the expression of IDO in cells are well known and include, but are not limited to, any of those described herein and those described, for example in U.S. Pat. Nos. 6,395,876; 6,451,840; and 6,482,416 and U.S. Published Appl. Nos. 2003/0194803; 2004/0234623; 2005/0186289 and 2006/0292618. IDO degrades the essential amino acid tryptophan.

Expression of IDO by human monocyte-derived macrophages and human dendritic cells allows these different antigen-presenting cells (APCs) to inhibit T cell proliferation in vitro. In vivo, IDO participates in maintaining maternal tolerance toward the antigenically foreign fetus during pregnancy.

The transfection of IDO into mouse tumor cell lines confers the ability to suppress T cell responses both in vitro and in vivo, and inhibition of IDO by administration of IDO inhibitors is capable to boost antitumor immunity with a concomitant antitumor effect. In a Lewis Lung carcinoma model, administration of 1-MT significantly delayed tumor outgrowth. The mouse mastocytoma tumor cell line P815 forms lethal tumors in naive hosts, but is normally rejected by pre-immunized hosts. However, transfection of P815 with IDO prevents its rejection by pre-immunized hosts. Inhibition of tumor growth was entirely dependent on the presence of an intact immune system and was substantially reversed, that is, tumor growth inhibited, by the concomitant administration of 1-MT.

The selective recruitment of IDO⁺ APCs in the tumor-draining (sentinel) lymph nodes of patients with melanoma indicates that tumors take advantage of the immunosuppressive effect of IDO by recruiting a population of IDO-expressing host APCs to present tumor antigens. Similar changes have been seen in breast carcinoma and other tumor-associated lymph nodes. In mouse tumor models the IDO-expressing APCs in tumor-draining lymph nodes are phenotypically similar to a subset of dendritic cells recently shown to mediate profound IDO-dependent immunosuppression in vivo IDO-expressing APCs in tumor-draining lymph nodes thus constitute a potent tolerogenic mechanism.

Plasmacytoid dendritic cells (PDCs) are a unique dendritic cell (DC) subset that plays a critical role in regulating innate and adaptive immune responses. PDCs sense the microbial pathogen components via Toll-like receptor (TLR) recognition, rapidly produce large amounts of type I interferons (including IFN-α and IFN-β), and activate diverse cell types such as natural killer (NK) cells, macrophages, and CD11c+DCs to mount immune responses against microbial infections. In addition to stimulating immune responses, increasing evidence suggests that PDCs may also represent a naturally occurring regulatory DC subset. Under certain circumstances PDCs appear to be able to induce the differentiation of regulatory T cells (Tregs) that downregulate immune responses. In humans, PDCs can prime allogeneic naive CD8+ T cells to differentiate into CD8+ suppressor T cells. It has recently been shown that human PDCs also induce the generation of CD4+ Tregs. These CD4+ Tregs strongly inhibit autologous or allogeneic T cell proliferation in vitro. Tregs are critical in maintaining self-tolerance and controlling excessive immune reactions, so their generation by PDCs is potentially of high biologic significance. However, the mechanism underlying PDC-induced CD4+ Treg generation remains unknown.

Tregs represent a critical barrier to immunotherapy of tumors. Established tumors suppress immune responses against their own antigens, and Tregs are emerging as a key mechanism contributing to this state of functional unresponsiveness. In murine models, host Tregs become activated within days of tumor implantation. Once activated, Tregs are difficult to eliminate, and serve to potently and dominantly inhibit otherwise effective immune responses against the tumor.

Tregs are potent suppressors of T cell mediated immunity in a range of inflammatory conditions, including infectious disease, autoimmunity, pregnancy and tumors. Mice lacking Tregs die rapidly of uncontrolled autoimmune disorders. In vivo, a small percentage of Tregs can control large numbers of activated effector T cells. Although freshly isolated Tregs exhibit minimal constitutive suppressor functions, ligating the T cell antigen receptor (TCR) in vitro or pre-immunizing mice with high-dose self-antigen in vivo stimulates Treg suppressor functions. This requirement for TCR signaling to enhance Treg suppressor functions is paradoxical because most Tregs are thought to recognize constitutively expressed self-antigens.

The CD4⁺ T cell lineage is emerging as more plastic that previously thought. In the case of Tregs, it is known that certain forms of inflammation or activated DCs can block Treg suppressive activity via a mechanism dependent at least in part on IL-6. Tregs that have been “de-activated” by such signals may downregulate Foxp3, and upregulate helper/effector cytokines such as IL-2 and IL-17. However, it has not been known whether this Treg plasticity was biologically relevant to tumor immunology, or whether it was amenable to therapeutic manipulation. The current invention demonstrates that widespread reprogramming of Tregs can occur physiologically in tumor-draining lymph nodes; that IDO is a key molecular regulator of this critically important checkpoint; and that this checkpoint can be pharmacologically targeted by an orally-bioavailable small-molecule inhibitor of IDO.

While Tregs can be suppressive, this is not a fixed and immutable attribute. Resting Tregs are not spontaneously suppressive, and require an activation step before they become functionally inhibitory. Conversely, the suppressive phenotype of Tregs is plastic. When Foxp3 is artificially ablated in mature Tregs, the suppressor phenotype is converted to a proinflammatory, T helper-like phenotype that can participate in autoimmunity. Likewise, Tregs exposed to certain inflammatory signals (e.g., from activated DCs or TLR ligands) can lose their suppressor activity, and may alter their phenotype (be “re-programmed”) to resemble proinflammatory effector cells. Thus, at least in these experimental models, Tregs show a significant degree of phenotypic plasticity, and are susceptible to both activation and de activation (reprogramming) by signals from their local microenvironment.

The current inventors have previously shown that Foxp3+ Tregs in the draining lymph nodes of mouse tumors become highly activated by exposure to the immunoregulatory enzyme indoleamine 2,3-dioxygenase (IDO). In tumor-draining lymph nodes (TDINs), IDO is expressed by a specific subset of IDO-competent plasmacytoid DCs. The combination of these IDO-expressing pDCs and IDO-activated Tregs renders the local milieu in the TDLN profoundly inhibitory for T cell activation.

### SUMMARY OF THE INVENTION

The current invention shows that under conditions of antigen-driven T cell response to tumors, IDO functions as a critical molecular “switch” in tumor-draining LNs, regulating the phenotype and functional activity of Tregs. Furthermore, the current invention demonstrates that when IDO is active, Tregs are maintained in their normal potently suppressive state; but when IDO is blocked, Tregs undergo an inflammation-induced, IL-6-dependent conversion into a non-suppressive, proinflammatory phenotype similar to TH17 cells. Additionally, the current invention shows that Tregs of the Foxp3 lineage constitute an integral part of the CD4⁺ T-helper system, and play a critical role in allowing innate inflammation to drive early (priming) phase of CD8⁺ T cell activation. Furthermore, the current invention shows that cross-presentation of a normal vaccine antigen to naïve CD8⁺ T cells is strongly dependent on reprogramming of local Tregs into helper cells, and that reprogrammed Tregs, but not conventional CD4⁺ T cells, are the main source of CD40L-mediated help during CD8⁺ T cell priming. Furthermore, the current invention surprisingly shows that the failure of therapeutic immunization in tumor-bearing hosts is because Treg programming is suppressed by tumor-induced IDO, and pharmacological inhibition of IDO enzymatic activity or pharmacological inhibition of the IDO pathway at the time of vaccination corrects this defect, thereby restoring Treg reprogramming and vaccine efficacy. These findings position IDO as a previously unsuspected key molecular regulator of Treg phenotype and function in TDLNs.

In one embodiment, the present invention relates to a method for reprogramming regulatory T cells (Treg) to acquire a pro-inflammatory T-helper-like phenotype comprising exposing said Treg cells to an effective amount of an inhibitor of indoleamine 2,3-dioxygenase or to an inhibitor of the IDO pathway. In a further embodiment, the Treg cells are also exposed to an effective amount of a vaccine. In yet a further embodiment, the inhibitor of IDO is 1-methyl tryptophan. In still a further embodiment, the inhibitor of the IDO pathway is 1-methyl-D-tryptophan (D1MT).

In another embodiment of the present invention, the Treg cells are exposed to an effective amount of a vaccine the vaccine comprises an antigenic protein or nucleic acid encoding an antigenic protein. In one embodiment, the vaccine is a viral vector. In a further embodiment the vaccine is a lentiviral vector. In still a further embodiment, the vaccine is a lentiviral vector that encodes a tumor antigen.

In another embodiment, the present invention relates to a method for reprogramming Treg cells to acquire a pro-inflammatory T-helper-like phenotype comprising exposing said Treg cells to an effective amount of a vaccine and D1MT and further exposing the Treg cells to IL-6. In one embodiment, the exposure to the vaccine, D1MT and/or IL-6 is performed in vitro. In another embodiment, the exposure to the vaccine, D1MT and/or IL-6 is performed in vivo.

In one embodiment, Treg cells are exposed to a vaccine prior to exposure to D1MT. In another embodiment, Treg cells are exposed to a vaccine concurrent with exposure to D1MT. In yet a further embodiment, Treg cells are exposed to vaccine after exposure to D1MT. In one embodiment, the D1MT is formulated for oral delivery. In a further embodiment, the D1MT is formulated as a powder, capsule tablet or liquid.

In another embodiment, the present invention relates to a method for reprogramming Treg cells to acquire a pro-inflammatory T-helper-like phenotype comprising exposing the Treg cells to an effective amount of a B7 ligand and an inhibitor of IDO. In a further embodiment the inhibitor of IDO is 1-methyl-tryptophan. In yet a further embodiment, the inhibitor of if IDO is 1-methyl-D-tryptophan. In a further embodiment, the B7 ligand is CD28-Ig.

In another embodiment, the present invention relates to a method for reprogramming Treg cells to acquire a pro-inflammatory T-helper-like phenotype comprising exposing the Tregs to a sufficient quantity of IL-6 and D1MT.

In another embodiment, the present invention relates to a method for increasing the immune response elicited by a vaccine comprising administering to a patient, a vaccine and D1 MT.

In another embodiment, the present invention relates to a method to induce conversion of FoxP3⁺ regulatory T cells into pro-inflammatory T-helper-like cells in an individual comprising administering to the individual a vaccine and an inhibitor of IDO. In a further embodiment, the inhibitor of IDO is D1MT. In still a further embodiment, the vaccine is an isolated protein administered in combination with an adjuvant. In one embodiment, the adjuvant is CpG oligonucleotides.

In another embodiment, the present invention relates to a method of treating a tumor in an individual comprising administering to the individual an anti-tumor vaccine and D1MT such that growth of the tumor is inhibited or reversed. In one embodiment, the anti-tumor vaccine is a lentiviral vaccine. In a further embodiment, the method further comprises administering IL-6 to the individual.

### BRIEF DESCRIPTION OF THE FIGURES

FIG. 1A shows that IDO-activated Tregs acquired efficient suppressor activity an order of magnitude more efficient on a per cell basis than the same Tregs activated using anti-CD3 antibodies plus IL2. FIG. 1B shows that antigen activated CD8⁺ cells must be present into order for Tregs to become activated by IDO. FIG. 1C shows that blocking IDO with D1MT prevents Tregs from acquiring suppressor activity.

FIG. 2A shows Tregs exposed to IDO⁺ PDCs and activated OT-I cells showed no IL-17 expression either when the cognate antigen OVA was absent or when the IDO pathway was not blocked by D1MTbut Tregs exposed to activated OT-I when IDO pathway was blocked by D1MT in the presence of the cognate antigen OVA contained a substantial proportion of cells that had upregulated IL-17, indicating conversion of the phenotype from Tregs to helper TH17 cells. FIG. 2B shows that Rorγt-null Tregs were unable to convert to IL-17 expression when the IDO pathway is blocked by D1MT. FIG. 2C shows that wild-type Tregs activated in the presence of D1MT lost all functional suppressor activity, whereas RORγt-deficient Tregs activated under identical conditions retained a significant suppressor activity even in the presence of D1 MT. FIG. 2D shows that IL-22 was co-expressed by essentially all of the Tregs that had upregulated IL-17. FIG. 2E shows that most of the re-programmed cells co-expressed IL-2 and TNFα in addition to IL-17 and IL-22 while only a small number expressed IFNγ or IL-10.

FIG. 3A shows that IL-6 was expressed PDCs only when IDO was blocked with D1MT. FIG. 3B shows that blocking IL-6 completely abrogated upregulation of IL-17 in Tregs in co-culture, even in the presence of D1MT. FIG. 3C shows that addition of exogenous recombinant IL-6 to co-cultures drove even more conversion of Tregs in the presence of D1MT, such that the large majority now became converted to the TH17-like phenotype. FIG. 3D shows that IL-6 induction in pDCs also required a signal from activated OT-1 cells and the presence of the cognate antigen in addition to blockade of the IDO pathway. FIG. 3D also shows that the signal from OT-I cells could also be replaced by recombinant CD28-Ig.

FIG. 4A diagrammatically depicts the IDO pathway whereby IDO depletes the amino acid tryptophan which can activate the amino-acid sensitive GCN2-kinase leading to reduced IL-6 production. FIG. 4B shows that blocking the IDO pathway in pDCs by either providing D1MT or by genetic ablation of either the IDO1 gene or the GCN2 gene leads to PDCs that are unable to suppress their own IL-6 production in co-cultures. FIG. 4C shows that both IDO1-KO and GCN2-KO PDCs spontaneously drove conversion of Tregs to TH17 like cells in co-cultures without the requirement for added D1MT. FIG. 4D shows that induction of the IDO gene triggered up-regulation of the inhibitory LIP isoform of NF-IL6, and that this was blocked by two different functional inhibitors of IDO enzymatic activity.

FIG. 5A shows that mice having B16-OVA tumors receiving only OT-I cells had no IL-17 expression by the endogenous Treg cells in TDLNs. Mice receiving OT-I plus concomitant D1MT administration showed a minority of Treg cells converting to IL-17 expression. Mice receiving OT-I plus vaccine (without D1MT) showed little IL-17 expression. However, the combination of a vaccine plus D1MT resulted in conversion of the majority of Tregs into IL-17 expressing cells. FIG. 5B shows that many of the pDCs in TDLNs upregulated IL-6 when challenged with OT-I cells in the presence of D1MT. FIG. 5C shows that in control recipients (without vaccination or 1MT) none of the transferred OT-II^{FoxP3-GFP-Thy}1.1 Tregs in TDLNs converted to IL-17 expression. However, in mice treated with OVA-Lv vaccine and 1MT, the majority of transferred Tregs in TDLNs upregulated IL-17. FIG. 5D shows that upregulation of IL-17 by Tregs in TDLNs also required an intact RORγt transcription factor in the Tregs.

FIG. 6A shows that mice with B16-OVA tumors which received adoptive transfer of OT-1, OVA-1v vaccine and cells D1MT, had significantly smaller tumors at day 11 compared with controls. This reduction in tumor volume is bigger than the sum of the individual effects OVA-Lv vaccine and D1MT. FIG. 6B shows that when followed for a longer period, tumors treated with D1MT plus OT-1 and vaccine showed a sustained growth delay. FIG. 6C shows that mice receiving D1MT alone showed few Tregs converting to IL-17 expression and mice receiving vaccine alone showed minimal conversion. However, mice receiving the combination of muTRP1-Lv vaccine and 1MT showed conversion of a large majority of Tregs in TDLNs into TH17-like cells. FIG. 6D shows that reprogramming of Tregs was associated with enhanced functional anti-tumor responses to muTRP1-Lv vaccine measured by tumor size on day 11. Again, this reduction in tumor volume is bigger than the sum of the individual effects muTRP1-Lv vaccine and D1MT.

FIG. 7A shows that a vaccine comprising OVA protein emulsified in incomplete Freund's adjuvant plus CpG oligodeoxynueleotide 1826 by itself had only a modest effect against established B16-OVA tumors, but that the addition of 1MT showed significant synergy with vaccine. FIG. 7B showed that in the tumors themselves, CEST-labeled OT-1 cells showed better ability to divide and upregulate differentiation markers (granzyme B and CXCR3) in mice treated with 1MT+Vaccine, compared to vaccine alone.

FIG. 8A shows analysis of CD4⁺ in the vaccine draining lymph node during the first 48 hours following immunization. FIG. 8B shows that prior to vaccination, resting Tregs from FoxP3^{GEP} mice produced no IL-2 or TNFα when challenged with PMA. However, after vaccination many Tregs had acquired the ability to produce IL-2 and TNFα, and large numbers also co-expressed IL-17. FIG. 8C shows that CD40L was upregulated on a subset of Tregs beginning at approximately 15 hrs after vaccination. FIG. 8C also shows that during the early phase of a priming immunization the only cells expressing CD40L were derived exclusively from the Treg population (GFP⁺), and there was no expression by conventional CD4⁺ T cells. FIG. 8D shows that immunization of these mice caused phenotypic alteration revealed by inducible cytokine expression, and also constitutive upregulation of CD40L, expression and this was confined exclusively to FoxP3⁺ Treg population. FIG. 81i shows that the acquisition of constitutive CD40L expression required the presence of CpG in the vaccine. FIG. 8F shows that Tregs adoptively transferred into hosts lacking MyD88 were unable to upregulate CD40L on Tregs in response to CpG vaccine.

FIG. 9A shows that TCRα-KO mice receiving no CD4⁺ cells or conventional CD4⁺ cells followed by OT-1 and vaccination with OVA, then OT-I cells showed little proliferation, poor cell recovery and no upregulation of granzyme B. However, adoptive transfer of Tregs provided effective help, driving robust OT-I proliferation and granzyme B upregulation. FIG. 9B shows that in TCRα-KO mice receiving no Tregs, the DCs in vaccine draining lump nodes were unable to upregulate CD80 and CD86 and that OT-I cells did not divide or express granzyme B. FIG. 9C shows that adoptive transfer of Tregs drove upregulation of costimulatory molecules on essentially all of the CD8α⁺ DCs, and on some of the CD8α^{NEG} DCs as well. FIG. 9D shows that the effect of Tregs can be replaced by injecting mice with an activating antibody against CD40, which mimics the effect of CD40L.

FIG. 10 demonstrates the progressive loss of T cell responsiveness to vaccination during B16F10 tumor growth.

FIG. 11A shows that mice with established tumors displayed impaired reprogramming of Tregs in tumor-draining LNs following vaccination. However, when IDO was blocked with D1MT, the same vaccination caused extensive Treg reprogramming. FIG. 11B shows experiments in which a cohort of Tregs (Thy1.2⁺) was enriched from either GCN2-KO mice or WT B6 mice controls and transferred into Thy1.1⁺ hosts. Mice then received tumors and were vaccinated but were not treated with 1MT. Under conditions in which IDO was active there was little detectable programming of the WT Treg cohort, but the Treg cohort from GCN2-KO mice were resistant to the effects of IDO and underwent normal reprogramming following vaccination despite the presence of tumor and without the need for 1MT. FIG. 11C shows that after vaccination, all of the CD4⁺ cells that expressed pro-inflammatory cytokines and CD40L derived exclusively from the original Treg population, whereas the non-Treg population contributed none of these cells.

FIG. 12A shows that responses of pmel-1 cells in tumor bearing hosts could be restored if IDO was blocked by treating mice with D1MT at the time of vaccination. FIG. 12B shows that the beneficial effect of D1MT on anti-tumor vaccination was strictly dependent on Treg-derived helper activity.

FIG. 13A shows that in resting control mice with no tumors, DCs expressed basal low levels of CD80 and CD86, but in mice with established tumors, this expression was almost completely lost following vaccination with 1 MT, however in mice treated with 1MT at the time of vaccination, DC expression of CD80 and CD86 was upregulated at high levels. FIG. 13B shows that only those mice receiving CD40L-sufficient Tregs, but not those receiving CD40L-KO-Tregs, were able to support full CD8⁺ T cell responses in the presence of D1MT. FIG. 13C shows that the defect in helper activity of CD40L-KO Tregs could be substantially rescued by treating mice with cross-linking anti-CD40 antibody.

### DEFINITIONS

As defined herein “vaccine” has a context dependent meaning. In the context of in vivo applications, or in the context of administration of a vaccine to an individual, a vaccine refers to any antigenic composition used to elicit an immune response. The antigenic composition can be unmodified peptides, glycosylated peptides, purified or recombinant proteins, viral vector vaccines or whole cells or cell fractions. A vaccine can be used therapeutically to ameliorate the symptoms of a disease, or prophylactically, to prevent the onset of a disease. In the context of exposing a Treg to a vaccine, the term “vaccine” refers to a “vaccine-derived peptide” or “TAA-derived peptide” as defined herein.

As used herein, the term “antigen” is meant any biological molecule (proteins, peptides, lipoproteins, glycans, glycoproteins) that is capable of eliciting an immune response against itself or portions thereof, including but not limited to, tumor associated antigens and viral, bacterial, parasitic and fungal antigens.

The term “Tumor Associated Antigens” or “FAA” refers to any protein or peptide expressed by tumor cells that is able to elicit an immune response in a subject, either spontaneously or after vaccination. TAAs comprise several classes of antigens: 1) Class I HLA restricted cancer testis antigens which are expressed normally in the testis or in some tumors but not in normal tissues, including but not limited to antigens from the MAGE, BACK, GAGF, NY-ESO and BORIS families; 2) Class I HLA restricted differentiation antigens, including but not limited to melanocyte differentiation antigens such as MART-1, gp100, PSA Tyrosinase, TRP-1 and TRP-2; 3) Class I HLA restricted widely expressed antigens, which are antigens expressed both in normal and tumor tissue though at different levels or altered translation products, including but not limited to CEA, HER2/neu, hTERT, MUC1, MUC2 and WT1; 4) Class I HLA restricted tumor specific antigens which are unique antigens that arise from mutations of normal genes including but not limited to β-catenin, α-fetoprotein, MUM, RAGE, SART, etc; 5) Class II HLA restricted antigens, which are antigens from the previous classes that are able to stimulate CD4+ T cell responses, including but not limited to member of the families of melanocyte differentiation antigens such as gp 100, MAGE, MART, MUC, NY-ESO, PSA, Tyrosinase; and 6) Fusion proteins, which are proteins created by chromosomal rearrangements such as deletions, translocations, inversions or duplications that result in a new protein expressed exclusively by the tumor cells, such as Bcr-Abl.

The term “TAA-derived peptides” or “vaccine-derived peptides” refer to amino acid sequences that bind to MI-IC (or HLA) class I or class II molecules. These peptides are amino acid sequences contained within proteins present in the vaccine, or contained within proteins which are encoded by a nucleic acid vaccine or viral vector vaccines such as a lentiviral vector vaccine encoding a tumor associated antigen. These peptides are generated in the individual receiving a vaccine containing or encoding such proteins, by the natural mechanisms of antigen uptake, processing and presentation carried out in antigen presenting cells such as PDCs, B cells, dendritic cells, and macrophages. Alternatively, these peptides can be chemically synthesized and used as a vaccine administered to an individual or used to expose a Treg in vitro.

### DETAILED DESCRIPTION OF TUE INVENTION

The present invention relates to methods of modulating Treg phenotype in vitro and in vivo. More specifically, the present invention concerns methods of inducing Treg conversion to helper cells via inhibition of IDO or the IDO pathway and thereby boosting efficacy of vaccine specific immune responses.

The present invention demonstrates a novel role for FoxP3⁺ Treg cells in the initial priming of CD8⁺ T cells to a cross-presented antigen. The present invention further shows that Tregs, via their ability to rapidly upregulate CD40L, form a mechanistic intermediate linking vaccine-induced inflammation with CD40L-mediated licensing of dendritic cells. The current invention also demonstrates that a large fraction of Tregs are constitutively ready to undergo rapid conversion to helper cells in response to innate inflammation and provide the required early help for CD8⁺ responses. Therefore, the present invention includes methods of enhancing the priming of vaccine antigen-specific CD8⁺ T cells via CD40-CD40L interactions. More specifically, the current invention includes methods of upregulating CD40L on reprogrammed Treg cells via inhibition of IDO and thereby enhancing the priming of vaccine antigen-specific CD8⁺ cells via CD40-CD40L interactions.

The present invention also demonstrates that Tregs in tumor draining lymph notes retain a remarkable degree of phenotypic plasticity. Under the right conditions, a large majority of Tregs in tumor draining lymph nodes could be reprogrammed in situ into a polyfunctional T-helper phenotype resembling inflammatory TH17 cells. Using in vitro and in vivo models, the current invention shows that this conversion requires signals from activated effector T cells, combined with inhibitions of the immunosuppressive IDO pathway. Therefore, the current invention includes methods of reprogramming Treg cells in tumor draining lymph nodes to TH17 cells via inhibition of the IDO pathway. In a particular embodiment, Tregs are reprogrammed in vivo or in vitro by administering an inhibitor of IDO the pathway. In a more preferred embodiment, the inhibitor of the IDO pathway is 1-methyl-D-tryptophan.

The current invention demonstrates that the phenotype of re-programmed Tregs was similar to activated TH17 cells or to “polyfunctional” T helper cells, since they co-express both IL-17 and IL-22, and also IL-2 and TNFα. These cells are herein referred to as “TH17-like” because of their RORγt-dependent induction of IL-17 expression. The current invention shows that these cells are a potent source of helper cytokines and play an indispensable role in the synergistic anti-tumor effect of 1 MT. Therefore, the current invention relates to a method of reprogramming Treg cells to IL-17, IL-22, IL-2 and TNFα, producing TH17 cells cells via inhibition of the IDO pathway.

The present invention also demonstrates that widespread reprogramming of Tregs can occur physiologically in tumor-draining LNs and that IDO is a key molecular regulator of this critically important checkpoint. Furthermore, the present invention shows that this check point can be pharmacologically targeted by an orally bioavailable small-molecule inhibitor of IDO. Therefore the current invention relates to methods of reprogramming Tregs in vivo in tumor draining lymph nodes by inhibiting IDO, for example by administering IDO inhibitors before during or after administration with a vaccine.

The observations of the present invention have wide applicability, including for example, in priming CD8⁺ T cells for treatment of bacterial and/or viral infection, vaccination, and cancer therapy. Additionally, according to the current invention, Tregs may be reprogrammed in vitro, for example, by exposing a population of Tregs to a vaccine antigen before during or after exposure to an IDO inhibitor. Therefore, the current invention includes methods of treating an individual in need thereof with an IDO inhibitor, in an amount sufficient to reprogram Tregs to TH17-like cells, before, during or after administration of a vaccine to the individual. The invention also includes methods of treating an individual in need thereof by administering Tregs which have been reprogrammed in vitro via IDO inhibition to TH-17-like cells, before, during or after administration of a vaccine to the individual.

Importantly, the current invention shows for the first time that signals from activated effector T cells are strictly required to drive conversion of Tregs to TH17-like cells when IDO is blocked. In vitro, this was shown by the requirement for antigen-activated CD8⁺ T cells (OT-I cells) in order to upregulate IL-6 in pDCs, and to drive conversion of Tregs. However, the signal supplied by antigen could be replaced by artificial ligation of B7 molecules using CD28-Ig fusion protein, suggesting that the role of activated antigen-specific CD8⁺ T cells was to provide a CD28→B7 mediated intracellular signal to the pDCs. The current invention therefore includes methods of upregulating IL-6 production by pDCs via B7 ligation in order to reprogram Tregs into TH17-like cells. In some embodiments, the B7 ligation occurs in vivo or in vitro via binding of CD28 on antigen-activated T cells. In other embodiments, the B7 ligation occurs in vito or in vitro via binding of CD28 Ig fusion protein.

In vivo, activated ovalbumin (OVA) specific T cells (OT-I) cells drives conversion of Tregs in TDLNs of mice with OVA-expressing tumors. Importantly, however, conversion of Tregs could also be driven by a vaccine against TRP1 (a shared self/tumor antigen) when combined with 1MT. The method of the current invention therefore involves the use of an immunogenic mutated TRP1 peptide capable of breaking tolerance to the native TRP1 protein, delivered in a lentivirus vaccine vector that stimulates robust CD8+ T cell responses. The efficacy of this vaccine in driving Treg conversion when combined with 1MT is an important finding, because it means that Treg conversion could be driven by the natural frequency of T cells against an endogenous self/tumor antigen, as long as IDO is blocked. Therefore, the current invention includes methods for driving the conversion of Tregs by blocking IDO activity and thereby enhancing the efficacy of vaccine-induced immune responses.

According to the current invention, conversion of Tregs to TH17-like cells required IL-6. Although other cytokines may also serve to bias cells toward the TH17 phenotype, neutralizing-antibody studies showed that IL-6 was strictly required according to the method of the current invention. In turn, the current invention shows that IL-6 expression was regulated by IDO, such that when IDO was active, production of IL-6 was suppressed. Thus, according to the current invention, a key molecular mechanism by which IDO maintains Tregs in the suppressive phenotype is by blocking the induction of IL-6 in activated pDCs. Therefore, the present invention includes methods of upregulating IL-6 production by inhibiting IDO and thereby driving the conversion of Tregs to TH17-like cells.

Taken together, according to the current invention, IDO functions as a molecular “switch” during certain forms of inflammation, acting to control the phenotype of local Tregs. Mice deficient in functional IDO do not show a global defect in Tregs, but they do show a profound defect in acquired peripheral tolerance, including acquired tolerance to transplanted tissues, fetal antigens, and antigens presented at mucosal surfaces. Since tumors represent a dramatic example of acquired tolerance to their own antigens, the regulatory role of IDO may be highly relevant in this context. Thus, the current invention includes methods of boosting anti-tumor immunity by blocking IDO and thereby driving conversion of Tregs to a helper T cell phenotype.

Clinically, according the current invention, instead of attempting to physically deplete Tregs, it is possible to reprogram Tregs in vitro or in vivo into proinflammatory T-helper/TH17-like cells. Accordingly, the combination of anti-tumor vaccination plus an IDO inhibitor drug is an effective strategy to de-activate and reprogram Tregs to enhance immunity to human tumors. Blocking the IDO pathway by administration of IDO inhibitors, GCN2 inhibitors or 1-methyl-D-tryptophan enhances the immune response elicited by vaccines of different kinds (nucleic acids, proteins, whole cells, viral particles, virus-like particles, peptides, with or without adjuvants), by triggering the conversion of FoxP3⁺ Tregs into proinflammatory TH17 T-helper-like T cells. Therefore, the current invention includes methods of enhancing the immune response induced by a vaccine by driving the conversion of FoxP3⁺ Tregs to TH17 T-helper-like T cells. In one embodiment, this conversion is achieved by administering IDO or GCN2 inhibitors prior to or concurrent with administration of a vaccine. In a particular embodiment, this is achieved by administration of 1-methyl-tryptophan prior to or concurrent with vaccine administration, such as for example, nucleic acid, whole cell, viral particle, virus-like particle or peptide vaccines. In some embodiments, the vaccine is given in conjunction with an adjuvant, for example a toll-like receptor activating agonist, such as CpG DNA, single stranded RNA, polyI:C or Complete Freund's Adjuvant. In a preferred embodiment, the conversion of Tregs to TH17-like cells is achieved by administration of 1-methyl-tryptophan, for example, 1-methyl-D-tryptophan, before. during or after a lentiviral vaccine in conjunction DNA is given.

As used herein, an IDO inhibitor is a substance or a pharmaceutically acceptable form of it that can directly inhibit the enzymatic activity of IDO either in a competitive, non-competitive, uncompetitive or mixed mechanism in such a way that the degradation of tryptophan to kynurenine is impaired by such substance. Preferably, an inhibitor of IDO has an IC₅₀ or Kᵢ of less than 100 μM. Examples of IDO inhibitors include but are not limited to any of a variety of commercially available IDO inhibitors, such as, 1-methyl-DL-tryptophan, b-(3-benzoluranyl)-DL-alanine, b-(3-benzo (b) thienyl)-DL-alanine, 5-bromo-DL-tryptophan, or any of the competitive and noncompetitive inhibitors of IDO discussed in Muller et al (Muller et al. 2005, Expert Opin Thr Targets; 9:831-849) or described in US Patent Applications Nos. 20090155311, 20060258719, 20070203140 (including, but not limited to various N-hydroxyguanidines compounds), 20070185165 (including, but not limited to, various N-hydroxyamidinoheterocycles compounds), 20070173524 (including, but not limited to, various brassilexin and brassinin derivatives), and 20070105907 (including, but not limited to, various brassilexin and brassinin derivatives), WO2009/1332238, WO 2004/094409, PCT/US2004/005154, WO/2006/005185 (naphtoquinones derivatives), PCT/CA2005/001087, Gaspari et al., 2006, J Med Chem; 49:684-92 (brassinin derivatives), Muller et al., 2005, Nat. Med; 11:312-319, Peterson et al., 1993, Med Chem Res; 3:473-482 (substituted beta-carbolines), Sono et al., 1989, Biochemistry; 28:5392-9, Sono et al., 1996, Chem Rev; 96:2841, and Vottero et al., 2006, Biotechnol J; 1:282-288.

As used herein, an IDO pathway inhibitor is a small molecule or a pharmaceutically acceptable form of it that does not directly inhibit the enzymatic activity of IDO with an IC₅₀ or Kᵢ lower than 100 μM in a competitive, non-competitive or uncompetitive manner, but that mimics the phenotypic or pharmacodynamic effects of inhibition of MO enzymatic activity. An example of an IDO pathway inhibitor is 1-methyl-D-tryptophan.

The present invention demonstrates that IDO expression is necessary for the generation of CD4+ Tregs and demonstrates that this effect can be pharmacologically reproduced by the addition of a metabolic breakdown product of tryptophan, or an analog of a metabolic breakdown product of tryptophan. Tryptophan is also referred to herein as “Tryp,” “tryp,” “Trp” or “trp.” IDO degrades the essential amino acid tryptophan (Trp) to kynurenine (KYN), which is then metabolized by other enzymes to subsequent metabolites along the KYN pathway. In certain embodiments, the metabolic breakdown product of tryptophan is L-kynurenine, kynurenic acid, anthranilic acid, 3-hydroxyanthranilic acid, quinolinic acid, or picolinic acid, and an analog of a metabolic breakdown product of tryptophan is an analog of L-kynurenine, kynurenic acid, anthranilic acid, 3-hydroxyanthranilic acid, quinolinic acid, or picolinic acid. A metabolic breakdown product of tryptophan, or an analog of a metabolic breakdown product of tryptophan, may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

The terms “agonist” and “agonistic,” as used herein, refer to or describe an agent that is capable of substantially inducing, promoting or enhancing TLR biological activity or TLR receptor activation or signaling. The terms “antagonist” or “antagonistic,” as used herein, refer to or describe an agent that is capable of substantially counteracting, reducing or inhibiting TLR biological activity or TLR receptor activation or signaling. As used herein, a TLR9 agonist refers to an agent that is capable of substantially inducing, promoting or enhancing TLR9 biological activity or TLR9 receptor activation or signaling. TLR9 is activated by unmethylated CpG-containing sequences, including those found in bacterial DNA or synthetic oligonucleotides (ODNs). A TLR9 agonist may be a preparation of microbial DNA, including, but not limited to, E. coli DNA, endotoxin free E. coli DNA, or endotoxin-free bacterial DNA from E. coli K12. A TLR9 agonist may be isolated from a bacterium, for example, separated from a bacterial source; synthetic, for example, produced by standard methods for chemical synthesis of polynucleotides; produced by standard recombinant methods, then isolated from a bacterial source; or a combination of the foregoing.

In preferred embodiments, a TLR9 agonist is a synthetic oligonucleotide containing unmethylated CpG motifs, also referred to herein as “a CpG-oligodeoxynueleotide,” “CpGODNs,” or “ODN.” A CpG-oligodeoxynucleotide TLR9 agonist includes a CpG motif. A CpG motif includes two bases to the 5′ and two bases to the 3′ side of the CpG dinucleotide. CpG-oligodeoxynucleotides may be produced by standard methods for chemical synthesis of polynucleotides. CpG-oligodeoxynucleotides may be purchased commercially, for example, from Coley Pharmaceuticals (Wellesley, Mass.), Axxora, LLC (San Diego, Calif.), or InVivogen, (San Diego, Calif.). A CpG-oligodeoxynucleotide TLR9 agonist may includes a wide range of DNA backbones, modifications and substitutions. In some aspects of the invention, a TLR9 agonist is a nucleic acid that includes the nucleotide sequence 5′ CC 3′. In some aspects of the invention, a TLR9 agonist is a nucleic acid that includes the nucleotide sequence 5′-purine-purine-cytosine-guanine-pyrimidine-pyrimidine-3′. In other aspects of the invention, a TLR9 agonist is a nucleic acid that includes the nucleotide sequence 5′-purine-TCG-pyrimidine-pyrimidine-3′. In some aspects of the invention, a TLR9 agonist is a nucleic acid that includes the nucleotide sequence 5′-(TGC)ₙ-3′, where n≧1. In other aspects of the invention, a TLR9 agonist is a nucleic acid that includes the sequence 5′-TCGNN-3′, where N is any nucleotide.

High-dose CpG administered intravenously to normal mice causes widespread up-regulation of IDO in splenic DCs and inducing rapid Treg activation, resulting in systemic immune-suppression and tolerance. This immunosuppressive effect is very different from what the literature teaches regarding CpG, which is universally considered an immune activator (since TLR ligands are classical adjuvant compounds). However, there is a markedly bimodal response to CpG, with low-dose treatment (<25 ug i.v.) producing immune activation but with high-dose treatment (>50 ug i.v.) causing unexpected collateral IDO upregulation and immune suppression. In order to achieve a positive (activating) immune response to this high-dose CpG, it is necessary to block IDO by co-administering an IDO inhibitor such as D1MT. Furthermore, in hosts with established tumors and many chronic infections, IDO is often already upregulated to high levels by the tumor or chronic infection (Vaccinating such hosts is so-called “therapeutic” vaccination). In the presence of this local disease-upregulated IDO expression, CpG (even at low dose) entirely fails to induce the desirable proinflammatory response that would be expected from the literature. In this setting of a therapeutic vaccine administered to a host with an established tumor or other IDO-upregulating condition, the adjuvant effect of CpG is only restored if IDO is blocked by co-administration of an IDO-inhibitor drug. Conversely, in normal hosts, without any pre-established IDO-upregulating tumor or chronic infection (i.e., hosts in which vaccination would be prophylactic), no IDO inhibition is required. Therefore, one aspect of the current invention involves determining the level of IDO expression in a subject to determining if IDO has been upregulated and administering to said subject a vaccine with or without and IDO inhibitor based on the level of IDO upregulation. Measurement of the level of IDO expression in patients can be achieved by standard methods know to a person of skill in the art. For example, IDO expression can be measured by immunohistochemistry on thin section tissue samples obtained from tumor or tumor-draining lymph node biopsies. Alternatively, the level of IDO expression can be measured through measurement of kynurenine and tryptophan plasma concentration and comparing the ratio of kynurenine to tryptophan to that seen in healthy patients.

With the methods of the present invention, a TLR agonist may be administered at a low dosage. In human subjects, a low dosage of a CpG agonist is about 30 mg or less. A low dosage of a CpG agonist may be about 25 mg or less. A low dosage of a CpG agonist may be about 20 mg or less. A low dosage of a CpG agonist may be about 15 mg or less. A low dosage of a CpG agonist may be about 10 mg or less. A low dosage of a CpG agonist may be about 5 mg or less. A low dosage of a CpG agonist may be about 1 mg or less. A low dosage of a CpG agonist may be about 0.5 mg or less. A low dosage of a CpG agonist may be a range of any of these dosages. For example, a low dosage of a CpG agonist may be from about 0.5 mg to about 30 mg. Such a low dosage may be administered, for example, when a TLR agonist is administered as a vaccine adjuvant. Such a low dosage may, for example, be administered subcutaneously, intradermal, or intratumoral.

With the methods of the present invention, a TLR agonist may be administered at a high dosage. In human subjects a high dosage is greater than 30 mg. A high dosage may, for example, be greater than about 30 mg, greater than about 50 mg, greater than about 75 mg, greater than about 100 mg, greater than about 125 mg, greater than about 150 mg, or more. A high dosage may be up to about 125 mg, up to about 250 mg, up to about 500 mg, or more. Such a high dosage maybe administered, for example, to induce an immunosuppressive effect. Such a high dosage may be administered systemically, including, for example, intravenously.

A TLR agonist may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

The methods of the present invention may also be administered to a patient receiving a vaccine. Such a vaccine may be an anti-viral vaccine, such as, for example, a vaccine against HIV, or a vaccine against tuberculosis or malaria. The vaccine may be a tumor vaccine, including, for example, a melanoma, prostate cancer, colorectal carcinoma, or multiple myeloma vaccine. Dendritic cells (DC) have the ability to stimulate primary T cell antitumor immune responses. Thus, a tumor vaccine may include dendritic cells. Dendritic cell vaccines may be prepared, for example, by pulsing autologous DCs derived from the subject with synthetic antigens, tumor lysates, tumor RNA, by transfection of DCs with tumor DNA, or by creating tumor cell/DC fusions. The vaccine may include one or more immunogenic peptides, for example, immunogenic HIV peptides, immunogenic tumor peptides, or immunogenic human cytomegalovirus peptides (such as those described in U.S. Pat. No. 6,251,399). The vaccine may include genetically modified cells, including genetically modified tumor cells or cell lines genetically modified to express granulocyte-macrophage stimulating factor (GM-CST). In some aspects of the invention, a vaccine may include an antigen that is the target of an autoimmune response.

Certain pathological conditions, such as parasitic infections, AIDS (caused by the human immunodeficiency virus (HIV)), hepatitis C and latent cytomegaloviral (CMV) infections, are extremely difficult to treat in part because the macrophages act as reservoirs for the infectious agent. Thus, even though the cells are infected with by a foreign pathogen, they are not recognized as foreign. Additionally, these pathogical conditions have each been shown to upregulate IDO expression. The methods of the present invention, for example by inhibiting IDO, may be used to treat such pathological conditions including, but not limited to, viral infections, infection with an intracellular parasite, and infection with an intracellular bacteria. Viral infections treated include, but are not limited to, infections with the human immunodeficiency virus (HIV), hepatitis C virus or cytomegalovirus (CMV). Intracellular bacterial infections treated include, but are not limited to infections with Mycobacterium leprae, Mycobacterium tuberculosis, Listeria monocytogenes, and Toxplasma gondii. Intracellular parasitic infections treated include, but are not limited to, Leishmania donovani, Leishmania tropica, Leishmania major, Leishmania aethiopica, Leishmania mexicana, Plasmodium falciparum, Plasmodium vivax, Plasmodium ovate, and Plasmodium malariae. The efficacy of treatment of an infection may be assessed by any of various parameters well known in the art. This includes, but is not limited to, a decrease in viral load, an increase in CD4⁺ T cell count, a decrease in opportunistic infections, eradication of chronic infection, and/or increased survival time.

Current experimental methods of cancer treatment include tumor vaccination protocols including the administration of tumor peptides or whole cell tumor vaccines with CpG ODNs as immunostimulatory adjuvants. Currently CpG ODNs have been utilized as an adjuvant along with a tumor vaccine. The present invention provides methods to enhance the immunostimulatory capacity of DCs to tumor antigens by co-administration of one or more inhibitors of IDO along with the administration of a TLR agonist, in an amount effective to suppress the induction or activation of Tregs. The present invention includes methods of treating cancer in a subject by administering to the subject an inhibitor of IDO in an amount effective to suppress the induction or Tregs. The present invention also includes methods of treating cancer in a subject by administering an inhibitor of IDO along with a TLR agonist, such as, for example, a CpG oligonucleotide and/or an inhibitor of GCN2 and/or additional therapeutic treatments in an amount effective to suppress the induction or Tregs. Additional therapeutic treatments include, but are not limited to, surgical resection, radiation therapy, chemotherapy, hormone therapy, anti-tumor vaccines, antibody based therapies, whole body irradiation, bone marrow transplantation, peripheral blood stem cell transplantation, and the administration of chemotherapeutic agents (also referred to herein as “antineoplastic chemotherapy agent”). Antineoplastic chemotherapy agents include, but are not limited to, cyclophosphamide, methotrexate, 5-fluorouracil, doxorubicin, vincristine, ifosfamide, cisplatin, gemcitabine, busulfan (also known as 1,4-butanediol dimethanesulfonate or BU), ara-C (also known as 1-beta-D-arabinofuranosylcytosine or cytarabine), adriamycin, mitomycin, cytoxan, methotrexate, paclitaxel, docetaxel, temozolamide and combinations thereof. Additional therapeutic agents include, for example, one or more cytokines, an antibiotic, antimicrobial agents, antiviral agents, such as AZT, ddI or ddC, and combinations thereof. The cytokines used include, but are not limited to, IL-1.alpha., IL-1.beta., IL-2, IL-3, IL-4, IL-6, IL-8, IL-9, 11, 10, IL-12, IL-18, IL-19, IL-20, IFN-α, IFN-β, IFN-γ, tumor necrosis factor (TNF), transforming growth factor-β (TGF-β), granulocyte colony stimulating factor (G-CSF), macrophage colony stimulating factor (M-CSF), granulocyte-macrophage colony stimulating factor (GM-CST), (See e.g. U.S. Pat. Nos. 5,478,556; 5, 837, 231 and 5,861,159) or Flt-3 ligand. Antitumor vaccines include, but are not limited to, peptide vaccines, whole cell vaccines, genetically modified whole cell vaccines, recombinant protein vaccines or vaccines based on expression of tumor associated antigens by recombinant viral vectors.

In hosts with established tumors, Treg reprogramming is suppressed by tumor induced indoleamine 2,3-dioxygenase (IDO) and vaccination failed due to lack of help. Reprogramming of Tregs, vaccine efficacy and anti-tumor CD8+ responses were restored by blocking IDO at the time of vaccination. Furthermore, in tumor bearing mice, the net effect of the IDO/GCN2 pathway was to dominantly inhibit Treg reprogramming after vaccination. It was this failure of Treg conversion, and the consequent loss of helper activity that was a major contributor to the failure of vaccination in hosts with established tumors. Blocking IDO pathway with D1MT rescued Treg reprogramming, and substantially restored the efficacy of vaccination. While this is certainly not the only pathway of immunosupresson in tumor-bearing hosts, it proved to be a critical and previously unappreciated checkpoint in our system, and it could be targeted with a clinically relevant drug (D1MT) which is currently in Phase I clinical trials. Therefore in tumor bearing hosts, the Tregs are not only highly suppressive they are also prevented from contributing their essential helper function after reprogramming. By blocking IDO at the time of immunization, Treg-mediated suppression could be converted into important helper activity.

The tumors to be treated by the present invention include, but are not limited to, melanoma, colon cancer, pancreatic cancer, breast cancer, prostate cancer, lung cancer, leukemia, lymphoma, sarcoma, ovarian cancer, Kaposi's sarcoma, Hodgkin's Disease, Non-Hodgkin's Lymphoma, multiple myeloma, neuroblastoma, rhabdomyosarcoma, primary thrombocytosis, primary macroglobulinemia, small-cell lung tumors, primary brain tumors, stomach cancer, malignant pancreatic insulinoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

The efficacy of treatment of a tumor may be assessed by any of various parameters well known in the art. This includes, but is not limited to, determinations of a reduction in tumor size, determinations of the inhibition of the growth, spread, invasiveness, vascularization, angiogenesis, and/or metastasis of a tumor, determinations of the inhibition of the growth, spread, invasiveness and/or vascularization of any metastatic lesions, and/or determinations of an increased delayed type hypersensitivity reaction to tumor antigen. The efficacy of treatment may also be assessed by the determination of a delay in relapse or a delay in tumor progression in the subject or by a determination of survival rate of the subject, for example, an increased survival rate at one or five years post treatment. As used herein, a relapse is the return of a tumor or neoplasm after its apparent cessation, for example, such as the return of leukemia.

The present invention includes the use of inhibitors of GCN2 to prevent the development or reactivation of Tregs by IDO. The protein kinase GCN2 (also referred to as “General Control Nonderepressible 2,” “eIF2AK4,” and “eukaryotic translation initiation factor 2 alpha kinase 4”) has been shown to play a role in the induction of proliferative arrest and anergy of CD8+ T cells in the presence of IDO+ DCs. Specifically, Munn et al. demonstrated that in order for IDO to mediate the proliferative arrest and anergy of effector T cells, the cells need GCN2. Thus, GCN2 is downstream in the pathway of IDO effects and inhibiting the function of GCN2 with an inhibitory agent should result in blockade of the inhibitory effect of IDO on the effector T cells. Thus, targeting GCN2 kinase with inhibitory agents can serve as an alternative to direct IDO inhibition. Thus, GCN2 has been implicated in mediating the effects of IDO in various cell types, including, but not limited to, effector CD8+ T cells and naive CD4+ T cells. Inhibitors of GCN2 may be used to bypass or replace the need for IDO inhibitors. The present invention includes any of the various methods described herein, in which an IDO inhibitor is replaced by or supplemented with a GCN2 inhibitor. Candidate GCN2 inhibitors, include, for example, a GCN2 blocking peptide, an antibody to GCN2 (both commercially available, for example, from Bethyl, Inc., Montgomery, Tex.), nucleic acid inhibitors such as siRNA, and small molecule inhibitors.

The present invention includes methods to enhance an immune response in a subject by administering an effective amount of an inhibitor of GCN2 kinase. With such a method a vaccine may also be administered, either simultaneously or shortly before or after the administration of an inhibitor of GCN2. The present invention includes methods to enhance the immune response in a subject to a vaccine antigen by administering to the subject the vaccine antigen, a CpG oligonucleotide (ODN), and an inhibitor of GCN2. The present invention also includes methods to enhance the immune response in a subject to a vaccine antigen by administering to the subject the vaccine antigen and an inhibitor of GCN2.

The present invention includes methods to prevent immune suppression mediated by Tregs with the administration of an effective amount of an inhibitor of a GCN2 kinase. The present invention also include methods to enhance an immune response in a subject by administering two or more agents selected from an inhibitor of indoleamine-2,3-dioxygenase (IDO), a CpG oligonucleotide (ODN), an inhibitor of GCN2 kinase, a vaccine, and/or a chemotherapeutic agent.

The present invention also includes methods to prevent immune suppression mediated by Tregs with the administration of two or more agents selected from an inhibitor of indoleamine-2,3-dioxygenase (IDO), a CpG oligonucleotide (ODN), an inhibitor of GCN2 kinase, a vaccine, and/or a chemotherapeutic agent.

The present invention includes compositions including one or more inhibitors of GCN2. In some embodiments, such a composition may also include one or more additional active agents, including, for example, one or more IDO inhibitors, one or more TLR agonists, such as, for example, one or more CpG oligonucleotides (ODN), one or more antigens, one o more metabolic breakdown products of tryptophan, one or more chemotherapeutic agents. Chemotherapeutic agents include, for example, an antineoplastic chemotherapy agent, including, but not limited to, cyclophosphamide, methotrexate, fluorouracil, doxorubicin, vincristine, ifosfamide, cisplatin, gemcytabine, busulfan (also known as 1,4 butanediol dimethanesulfonate or BU), araC (also known as 1-beta-D-arabinofuranosylcytosine or cytabine), adriamycin, mitomycin, cytoxan, methotrexate, or combinations thereof. Additional therapeutic agents include cytokines, including, but not limited to macrophage colony stimulating factor, interferon gamma, flt-3, an antibiotic, antimicrobial agents, antiviral agents, such as AZT, ddI, ddC or combinations thereof.

As used herein “treating” or “treatment” includes both therapeutic and prophylactic treatments. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis.

The agents of the present invention can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical, or injection into or around the tumor.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intraperitoneal, and intratumoral administration. In this connection, sterile aqueous media that can be employed will be known to those of skill in the art in light of the present disclosure (see for example, “Remington's Pharmaceutical Sciences” 15th Edition). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA.

For enteral administration, the inhibitor may be administered in a tablet or capsule, which may be enteric coated, or in a formulation for controlled or sustained release. Many suitable formulations are known, including polymeric or protein microparticles encapsulating drug to be released, ointments, gels, or solutions which can be used topically or locally to administer drug, and even patches, which provide controlled release over a prolonged period of time. These can also take the form of implants. Such an implant may be implanted within the tumor.

Therapeutically effective concentrations and amounts may be determined for each application herein empirically by testing the compounds in known in vitro and in vivo systems, such as those described herein, dosages for humans or other animals may then be extrapolated therefrom.

An agent of the present invention may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

With the present invention, the stimulation or inhibition of an immune response may be measured by any of many standard methods well known in the immunological arts. As used herein, a mixed leukocyte response (MLR) is a well-known immunological procedure, for example, as described in the examples herein. As used herein, T cell activation by an antigen-presenting cell is measured by standard methods well known in the immunological arts. As used herein, a reversal or decrease in the immunosuppressed state in a subject is as determined by established clinical standards. As used herein, the improved treatment of an infection is as determined by established clinical standards. The determination of immunosuppression mediated by an antigen presenting cell expressing indoleamine-2,3-dioxygenase (IDO) includes the various methods as described in the examples herein.

With the methods of the present invention, the efficacy of the administration of one or more agents may be assessed by any of a variety of parameters well known in the art. This includes, for example, determinations of an increase in the delayed type hypersensitivity reaction to tumor antigen, determinations of a delay in the time to relapse of the post-treatment malignancy, determinations of an increase in relapse-free survival time, determinations of an increase in post-treatment survival, determination of tumor size, determination of the number of reactive T cells that are activated upon exposure to the vaccinating antigens by a number of methods including ELISPOT, FACS analysis, cytokine release, or T cell proliferation assays.

As used herein, the term “subject” includes, but is not limited to, humans and non-human vertebrates. Non-human vertebrates include livestock animals, companion animals, and laboratory animals. Non-human subjects also include non-human primates as well as rodents, such as, but not limited to, a rat or a mouse. Non-human subjects also include, without limitation, chickens, horses, cows, pigs, goats, dogs, eats, guinea pigs, hamsters, mink, and rabbits. As used herein, the terms “subject,” “individual,” “patient,” and “host” are used interchangeably. In preferred embodiments, a subject is a mammal, particularly a human.

As used herein “in vitro” is in cell culture and “in vivo” is within the body of a subject.

As used herein, the term “pharmaceutically acceptable carrier” refers to one or more compatible solid or liquid filler, diluents or encapsulating substances which are suitable for administration to a human or other vertebrate animal.

As used herein, the term “isolated” as used to describe a compound shall mean removed from the natural environment in which the compound occurs in nature. For example, in one embodiment an isolated nucleic acid means that the nucleic acid is removed from non-nucleic acid molecules of a cell.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

In some embodiments, an “effective amount” of an agent is an amount that results in a reduction of at least one pathological parameter. Thus, for example, in some aspects of the present invention, an effective amount is an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter in an individual not treated with the agent.

The present invention is illustrated by the following examples. It is to be understood that the particular examples, materials, amounts, and procedures are to be interpreted broadly in accordance with the scope and spirit of the invention as set forth herein.

### EXAMPLES

### Methods

Reagents, 1MT, and cell lines

1-methyl-D-tryptophan (catalog #452483) and 1-methyl-L-tryptophan (catalog #447439) were purchased from Sigma (St. Louis Mo.) or supplied by NewLink Genetics Corporation (Ames, Iowa) or National Cancer institute (Rockville, Md.) and dissolved as described (Munn, et al. (2005) “GCN2 kinase in T cells mediates proliferative arrest and anergy induction in response to indoleamine 2,3-dioxygenase,” Immunity, 22:633-642). For in vivo use, D1MT in drinking water at 2 mg/ml (or vehicle control) was administered as described (Sharma, et al. (2007) “Plasmacytoid dendritic cells from mouse tumor-draining lymph nodes directly active mature T regs via indoleamine-2,3-dioxygenase,” J. Clin. Invest., 117:2570-2582).

Conjugated antibodies against mouse CD4, CD8, IL-10, Thy1.1, TNF, and CD11c were from BD-Pharmingen; antibodies against IL-17A, granzyme B, IL-2, and IL-6 were from eBioseience; antibodies against IL 22 and CXCR3 were from R&D Systems. Recombinant mouse IL-6 (R&D Systems) was used at 100 μg/ml. Polyclonal anti-mouse IL-6 antibody (cat. #AF-406-NA, R&D Systems) was used at 100 μg/ml. Recombinant mouse CD28/Fc chimeric protein (also known as CD28-Ig) (#483-CD, R&D Systems) was used at 20 μg/ml.

Cell lines used were B16F10 (Nicolson, et al. (1978) “Specificity of arrest, survival, and growth of selected metastatic variant cell lines,” Cancer Res., 38:4105-4111) (obtained from ATCC, Manassas, Va.) and B16 OVA (B16F10 transfected with full-length chicken ovalbumin, clone MO4 (Falo, et al. (1995) “Targeting antigen into the phagocytic pathway in vivo induces protective tumour immunity, Nat. Med., 1:649-653), obtained from Dr. Alan Houghton, Memorial Sloan Kettering).

Mouse Strains and Radiation Chimeras

TCR-transgenic OT-I mice (CD8+, B6 background, recognizing the SIINFEKL peptide of ovalbumin on H2Kb) were purchased from Jackson Laboratories (Bar Harbor, Me.). GCN2 KO mice (Munn, et al. (2005) “GCN2 kinase in T cells mediates proliferative arrest and anergy induction in response to indoleamine 2,3-dioxygenase,” Immunity, 22:633-642) (136 background) were a generous gill: from the laboratory of David Ron, New York University School of Medicine. Foxp3GFP mice (Fontenot, et al. (2005) “Regulatory T cell lineage specification by the forkhead transcription factor, foxp3” Immunity, 22:329-341) we re the generous gift of Alexander Rudensky and were inbred >5 generations onto the B6 background. Rorγtgfp/gfp mice (Eberl, et al. (2004) “An essential function for the nuclear receptor RORgamma(t) in the generation of fetal lymphoid tissue inducer cells,” Nat. Immunol., 5:64-73) were the generous gift of Dan Littman, New York University. A1 mice (CBA background, recognizing an H Y peptide presented on IEk) and IDO1 KO mice (B6 background) were as described (Sharma, et al., (2007) “Plasmacytoid dendritic cells from mouse tumor-draining lymph nodes directly active mature T regs via indoleamine-2,3-dioxygenase,” J. Clin. Invest., 117:2570-2582).

For Treg adoptive transfer, Tregs were isolated from spleens of TCR-tg OT-11 mice bred onto the Foxp3^{GFP} background. (Fontenot, et al. (2005) “Regulatory T cell lineage specification by the forkhead transcription factor, foxp3” Immunity, 22:329-341). OT-11 mice were obtained from Jackson and crossed to Foxp3GFP mice on a Thy1.1-congenic background. OT-11 mice possess Tregs which can respond to cognate OVA antigen (Sutmuller et al. (2006) “Toll-like receptor 2 controls expansion and function of regulatory T cells,” J. Clin. Invest. (116:485-494) and to B16 OVA tumors (Wang, et al. (2008) “Programmed death 1 ligand signalling regulates the generation of adaptive Foxp2⁺CD4⁺ regulatory T cells,” Proc. Natl. Acad. Sci. USA; 105:9331-9336), naive OT-II T cells are capable of differentiation into both Tregs and TH17 cells under appropriate conditions (Mucida, et al. (2007) “Reciprocal TH17 and regulatory T cell differentiation mediated by retinoic acid,” Science, 317:256-260).

For radiation chimeras, wt B6 recipients were irradiated (9.5 Gy) and transplanted with 1×10⁷ nucleated bone-marrow cells from either RORγt^{gfp/gfp} mice or wt B6 controls. Chimeras were used 8 weeks after transplant. IA^{b}-KO mice lack an IA^{b} promoter-exon 1 region due to Cre/loxP mediated deletion and were generated as described (Shimoda, et al. (2006) “Conditional ablation of MHC-II suggests an indirect role for in regulatory Cd4 T cell maintenance,”0.1. Immunol., 176:6503-6511) by crossing mice carrying a floxed 1A^{b} allele with TIE2-Cre mice (both on C57BL/6J background). The resultant heterozygous mice were interbred to establish an IA^{b}-KO breeding colony.

Mice transgenic for an EGFP-cre fusion protein under the Foxp3 promoter, derived by Bluestone and colleagues (Zhou et al., (2009) “Instability of the transcription factor Foxp3 leads to the generation of pathogenic memory T cells in vivo,” Nat. Immunol. Zhou et al., “2008) “Selective miRNA disruption in T reg cells leads to uncontrolled autoimmunity,” J Exp. Med., 205:1983-1991). were obtained from Jackson Laboratories (NOD/Shilt-Tg(Foxp3-EGFP/cre)1Jbs/J). These mice were crossed with reporter mice transgenic for YFP behind a floxed stop codon under the ROSA26 promoter (CBy.B6-Gt(ROSA)26Sor^{iml(HBEGF)Awai}/J, Jackson) (Srinivas et al., (2001) “Cre reporter strains produced by targeted insertion of EYFP and ECFP into the ROSA26 locus,” BMC Dev. Biol., 1:4.). F1 (double-hemizygous) offspring were used for all experiments. (The Foxp3-EGFP-cre mice were on the NOD background, but the mixed F1(NOD×B6) offspring were not diabetes-prone, and the F1 background was immaterial for our vaccination studies, because F1 mice had one full B6 haplotype and so could present antigen to OT-I cells, and there was no potential for host-versus-graft reaction.)

Tumors

Tumor implantation and harvesting of TDLNs are described in detail in Sharma et al 2007. A large inoculum of B16F10 and B 16-OVA tumor cells was used (1×10⁶) to ensure that established tumors rapidly drove Treg activation and suppression in the MIN. Tumor area was measured at necropsy on day 11 as the product of orthogonal diameters; or was measured serially 3×/week.

Vaccines

Recombinant lentivector expressing truncated cytoplasmic chicken ovalbumin (OVA Lv) was prepared by using transient co-transfection method as described. (He, et al. (2006) “Skin-derived dendritic cells induce potent CD8(+) T cell immunity in recombinant lentivector-mediated genetic immunization,” Immunity, 24:643-656). Plasmid DNA containing the mutant TRP1 gene (muTRP1) has been previously described (Guevara-Patino, et al. (2006) “Optimization of self antigen for presentation against tumor antigen-derived peptide,” J. Immunol., 116:1382-1390) and was the generous gift of Jose Guevara-Patino. Lentivector vaccines were delivered by footpad injection 5 days after tumor implantation, timed such that maximal production of antigen would coincide with the OT-I injection on day 7. CpG vaccines were prepared by emulsifying 100 ug OVA protein (Sigma F-5506) and 50 ug CpG 1826 (gift of Coley Pharmaceuticals) in incomplete Freund's adjuvant (Sigma) as described (Miconnet, et al. (2002) “CpG are efficient adjuvants for specific CTL induction against tumor antigen-derived peptide,” J. Immunol., 168:1212-1218) and administered in the footpad on day 7 at the time of OT-I transfer.

CpG-1826 (phosphorothioate oligo 5′-TCCATGACGTFCCTGAGCTT-3′) was synthesized based on the published sequence (Chu et al., 1997) by Tri-link Biotechnologies. All experiments shown used CpG-1826, but Treg reprogramming was a generalizable phenomenon, and similar results were seen with CpG-2359, and also with lentivirus-based vaccines in place of CpG. Human gp100₂₅₋₃₃ (KVPRNQDWL) was synthesized by Southern Biotechnology; this peptide functions as an altered peptide ligand for pmel-1 (Overwijk et al., (2003) “Tumor regression and autoimmunity after reverssal of a functionally tolerant state of self-reactive Cd8+ T cells.” J. Exp Med., 198:569-580). Whole OVA protein was obtained from Sigma (A-5503). Vaccines were prepared by emulsifying 100 ug of OVA protein, or 25 ug gp100 peptide, with 50 ug CpG 1826 in incomplete Freund's adjuvant (Sigma F-5506) and administered in the footpad.

Adoptive Transfer

For OT-I adoptive transfer, mice received 2×10⁶ sorted CD8+ OT-I spleen cells i.v (Sharma, et al. (2007) “Plasmacytoid dendritic cells from mouse tumor draining lymph nodes directly activate mature Tregs via indoleamine 2,3-dioxygenase,” J. Clin., 117:2570-2582). For Treg adoptive transfer, Tregs were isolated from spleens of TCR-tg OT-11 mice bred onto the Foxp3^{GFP} (Thy 1.1-congenic) background (Fontenot, et al. (2005) “Regulatory T cell lineaage specification by the forkhead transcription factor foxp3,” Immunity, 22:329-341), and FACS-sorted as CD4+GFP+ cells. OT-IIFoxp3^{GFP} Thy1.1 Tregs (1×10⁶) were mixed with OT-I cells for co-adoptive transfer.

Hosts were either C57BL/6, Foxp3^{GFP}, TCRαKO, or other KO mice as described in each experiment. For CD8⁺ T cell transfers, OT-I or pmel-1 spleen cells were enriched for CD8+ cells by magnetic bead isolation (Miltenyi Biotech), labeled with CFSE as described (Munn et al., (2005) “ ” GCN2 kinase in T cells mediates proliferative arrest and anergy in induction in response to indoleamine, 2,3-dioxygenase,” Immunity, 22:633-642), and 2×10⁶ cells injected i.v. All CD4⁺ transfers were sorted from spleens of Foxp3^{GFP} mice by MoFlo cell-sorter with doublet discrimination (>95% post-sort purity). Tregs (CD4⁺GFP⁺, 2×10⁵ cells) or non-Tregs (CD4⁺GFP^{NEG}, 1×10⁶ cells) were injected i.v. When F1(Foxp3-GFP-cre×ROSA-YFP) mice were used, Tregs were isolated based on the combined fluorescence in the FL1 (FITC) channel (both GFP⁺ and YFP⁺ signal), since the goal was to include all Foxp3-lineage cells. For CD40L-KO and GCN2-KO mice, which did not have a GFP transgene, Tregs were enriched by sorting for CD4⁺CD25⁺ cells (>90% Foxp3 cells by intracellular staining). In these experiments, the small number of contaminating non-Tregs did not affect the conclusions. (However, all experiments that used the non-Treg, conventional fraction of CD4⁺ cells were always sorted based on Foxp3^{GFP} fluorescence, to rigorously exclude all Tregs.)

FACS Staining

For intracellular cytokine staining, cells were harvested from co-cultures, or isolated from mechanically disaggregated TDLNs ex vivo, and incubated for 4 hrs with 5 ng/ml PMA+2 uM ionomycin (Bettelli, et al. (2006) “Reciprocal developmental pathways for the generation of pathogenic effector TH17 and regulatory T cells,” Nature, 441:235-238) in the presence of brefeldin A (GolgiPlug, BD Bioscience), then fixed in Cytoperm/Cytofix (BD Bioscience) on ice and stained in BD Permwash solution per the manufacturer's instructions. For tumor-disaggregation studies, tumors were treated for 1 hr with 1 mg/ml collagenase (Sigma C5138), 0.1 mg/ml DNAse (Sigma D5025) and 0.1 mg/ml hyaluronidase (Sigma H3884) in RPMI 1640 medium.

Treg Activation Co-Cultures and Readout Assays

The Treg culture system has been described in detail (Sharma, et al. (2007) “Plasmacytoid dendritic cells from mouse tumor-draining lymph nodes directly activate mature Tregs via indoleamine 2,3-dioxygenase,” J. Clin. Invest., 117:2570-2582). Pre-activation cultures contained 2×10³ pDCs, isolated by sorting for the CD11c+B220+ fraction, which contains the IDO-expressing subset of CD19+pDCs, as previously described. (Munn, et al. (2004) “Expression of indoleamine 2,3-dioxygenase by plasmacytoid dendritic cells in tumor-draining lymph nodes,” J. Clin. Invest., 114:280-290). To these were added 1×10⁵ sorted CD8+ OT-I cells, 100 nM SIINFEKL peptide, and 5×103 sorted CD4+CD25+Tregs from spleens of nom-tumor-bearing B6 mice, or CD4+GFP+Tregs from Foxp3C1FP mice. All cultures received a feeder layer of 1×10⁵ T-cell—depleted B6 spleen cells (CD4NEGCD8NEG), as described, (Sharma, et al. (2007) “Plasmacytoid dendritic cells from mouse tumor-draining lymph nodes directly activate mature Tregs via indoleamine 2,3-dioxygenase,” J. Clin. Invest., 117:2570-2582) in order to maintain viability of the sorted cell populations. We have previously shown that these feeder cells are entirely nonspecific, and can be MHC-mismatched or IDO-deficient. (Sharma, et al. (2007) “Plasmacytoid dendritic cells from mouse tumor-draining lymph nodes directly activate mature Tregs via indoleamine 2,3-dioxygenase,” J. Clin. Invest., 117:2570-2582). For αCD3-induced activation, identical cultures received 200 uM D1MT plus 0.1 ug/ml αCD3 mAb and 10 ng/ml IL-2. Cultures were harvested after 2 days, and either stained for FACS analysis, or the Tregs re-stained for CD4 expression and sorted for functional suppression assays (comprising 1×10⁵ A1 cells, 2×10³ CD11c+DCs from CRA spleen. and 11 Y peptide). Proliferation of readout assays was measured after 72 hrs by thymidine incorporation. An allogeneic readout was used to prevent any activation of the Tregs during the readout assay, so that suppression of the readout T cells was strictly dependent on activation of the Tregs in the pre-activation cultures.

Transfection of T-REX Cells with IDO and Western Blot for NF IL6

Western analysis of the LIP isoform of NF-IL6/CEBPβ was performed as described (Metz, et al. (2007) “Novel tryptophan catabolic enzyme IDO2 is the preferred biochemical target of the antitumor indoleamine 2,3-dioxygenase inhibitory compound D-1-methyl-tryptophan,” Cancer Res., 67:7082-7087) using equal amounts of protein from lysates derived from T-REX cells stably transfected with an inducible IDO construct (pcDNATO4-IDO, as described 15). Cells were seeded into 12 well dishes. IDO was not expressed in uninduced cells, and could be induced by treatment with Doxycycline (20 ng/mL). Replicate cultures of cells induced with Doxycycline were also treated with 50, 25, and 10 μM of the IDO enzyme inhibitors L-1MT or MTH-tryptophan (Muller, et al. (2005) “Inhibition or indoleamine 2,3-dioxygenase, an immunoregulatory target of the cancer suppression gene potentiates cancer chemotherapy,” Nat. Med., 11:312-319). To confirm functional IDO expression, cells were harvested 48 hours following treatment and lysed in RIPA buffer. Kynurenine production in cells was analyzed essentially as described (Muller, et al. (2005) “Inhibition of indoleamine 2,3-dioxygenase, an immunoregulatory target of the cancer suppression gene Bin1, potentiates cancer chemotherapy,” Nat. Med., 11:312-319). Briefly, 200 μL of the media form the treated cells was mixed with 12.5 μL 30% TCA, incubated 30 min at 50° C., and clarified by 10 min centrifugation at 3-10,000 rpm. Supernatants (100 μL) were removed to a new dish, mixed with 100 μL Ehrlich's reagent (2% p-dimethylaminobenzaldehydc w/v in glacial acetic acid), and incubated 10-30 min at room temperature. Absorbance was determined at 490 nm.

To confirm IDO induction in transfected cells by western blot, affinity purified rabbit polyclonal anti-IDO was prepared by a commercial supplier (Covance). Antisera was raised against a mixture of murine and human GST-conjugated MO (Munn, et al. (2005) “GCN2 kinase in T cells mediates proliferative arrest and anergy induction in response to indoleamine 2,3-dioxygenase, Immunity, 22:633-642). Antisera was screened for reactivity against the immunizing antigen by ELISA and western, and samples with high titer were purified by affinity chromatography. Specifically, antisera was preabsorbed to protein column containing GST and GST-IDO2 (and IDO related protein) The unbound material was then affinity purified on an antigen specific peptide column containing human and mouse His-tagged IDO1. The resulting antibody was analyzed and determine to be IDO1 specific with no cross-reactivity with IDO2. The primary antibody was detected using HRP-conjugated goat anti-rabbit antibody and chemiluminescence.

### Example 1

### IDO Plus Effector T Cells Activate Foxp3⁺ Tregs for Suppression

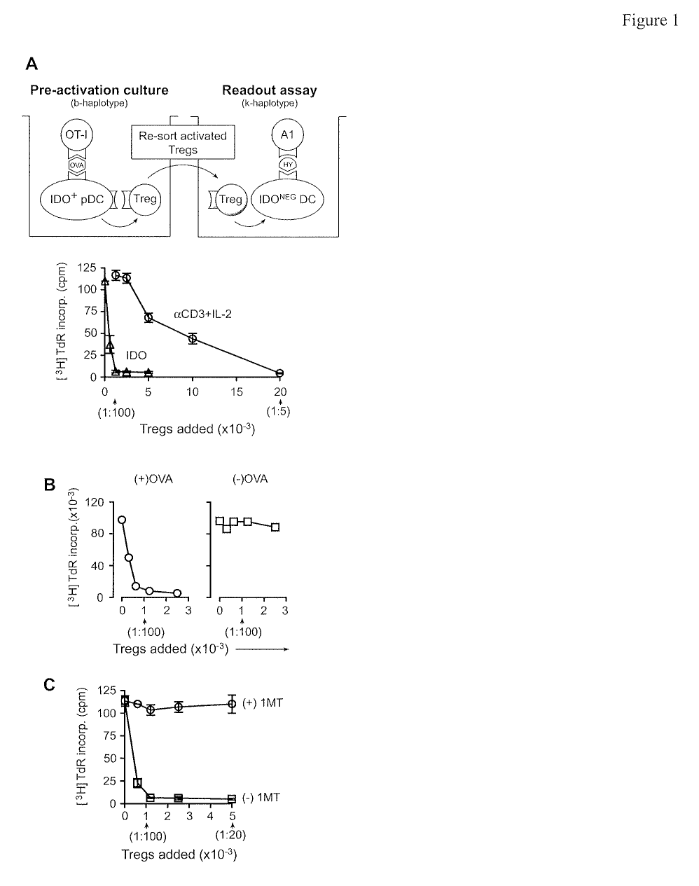

In vitro studies were performed using the co-culture system shown in FIG. 1A (described in Methods, from ref (Sharma, et al., (2007) “Plasmacytoid dendritic cells from mouse tumor-draining lymph nodes directly activate mature Tregs via indoleamine, 2-3-dioxygenase,” J. Clin. Invest., 117:1147-1154)). Resting Tregs (CD4+ CD25+) were sorted from spleens of B6 mice without tumors. IDO-expressing pDCs were enriched (CD11c+B220+) from the TDLNs of mice with B16 melanoma tumors. As a source of activated effector T cells, OVA-specific OT-1 T cells (sorted CD8 f) were added to co-cultures with cognate OVA peptide antigen. After 2 days, Tregs were recovered from co-cultures by FACS sorting and tested for suppressor activity in a readout assay comprising allogeneic A1 T cells (TCR-tg, recognizing a peptide of HY) plus congenic CBA spleen DCs.

FIG. 1A shows that IDO-activated Tregs acquired efficient suppressor activity. comparable to the most potent suppression reported in the literature (McHugh et al., 2002; Immunity, 16:311-323 and Caramelho, et al., 2003; J Exp Med, 197:403-411) and an order of magnitude more efficient on a per-cell basis than the same Tregs activated using anti CD3 antibodies plus recombinant IL-2 (FIG. 1A). (For CD3-induced activation, IDO was blocked by adding the IDO-inhibitor D1MT). We have previously shown that similar IDO-induced Treg activation also occurs in vivo in TDLNs (Sharma, et al., (2007) “Plasmacytoid dendritic cells from mouse tumor-draining lymph nodes directly activate mature Tregs via indoleamine, 2-3-dioxygenase,” Clin. Invest., 117:1147-1154).

In order for Tregs to become activated by IDO, it was also necessary for antigen-activated OT-I cells to be present. If the cognate OVA antigen for OT-I was omitted, then Tregs failed to acquire suppressor activity (FIG. 1B). Blocking IDO with D1MT also prevented Tregs from acquiring suppressor activity (FIG. 1C). Thus, two conditions were required for Tregs to become activated for suppression: functional IDO and activated OT-I.

### Example 2

### In the Absence of IDO, Tregs Undergo Conversion to a TH17-Like Phenotype

A key point not elucidated by the preceding experiments was the fate of those Tregs exposed to activated OT-I cells but without the signal from IDO. It is known that under certain proinflammatory conditions Tregs can lose their suppressor phenotype. T helper 17 (TH17) cells bear a reciprocal developmental relationship to inducible Tregs and some Tregs that lose their suppressive phenotype may upregulate IL-17. Therefore, we asked whether Tregs exposed to activated OT-I in the absence of IDO might convert to a phenotype resembling TH17 cells.

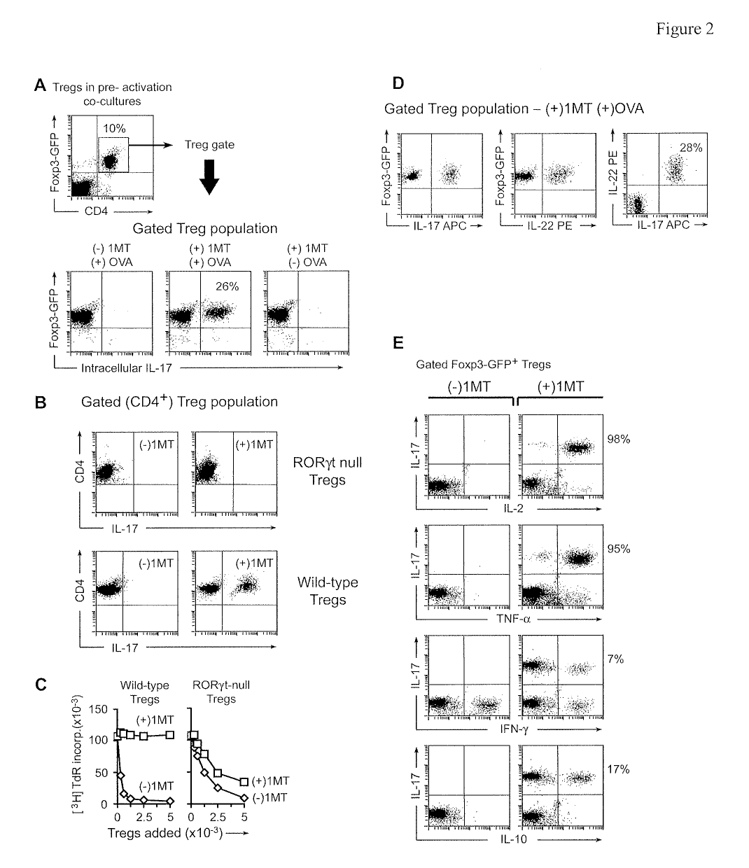

Tregs were FACS-sorted from mice bearing a Foxp3-GFP fusion protein in place of one normal Foxp3 gene. Sorted CD4+GFP+ cells from these mice were thus unambiguously known to be Foxp3+ Tregs at the start of the assay. Pre-activation co-cultures were performed as in FIG. 1, with or without OVA and D1MT. After 2 days co-cultures were harvested and stained for intracellular IL-17; FIG. 2A shows the gated Treg population (CD4+ Foxp3-GFP+). Tregs exposed to activated OT-I cells when IDO was active (no 1MT) showed no IL-17 expression; but Tregs exposed to activated OT-I when IDO effect was blocked by D1MT contained a substantial proportion of CD4+GFP+ cells that had upregulated IL-17. IL-17 upregulation required OT-I activation, since IL-17 was not induced in the absence of OVA antigen (last panel of FIG. 2A).

Of note, the IL-17-expressing cells in co-cultures were known to arise specifically from conversion of pre-existing Foxp3+ Tregs (not from differentiation of naive CD4+ cells), because the only CD4+ cells present in co cultures were the original Foxp3^{GFP}-positive Tregs. Furthermore, the newly-converted IL-17+ cells uniformly continued to express residual Foxp3^{GFP} fluorescence, confirming their origin from the original Foxp3+Tregs. (Similar co-expression of Foxp3 and IL-17 during Treg reprogramming has been observed in other systems as well (Osorio, et al 2008; Eur J Immunol, 38:3274-3281 and Yang, et al., 2008; “Immunity, 29:44-56.)

The transcription factor RORγt is required for normal differentiation of naive CD4− T cells along the TH17 lineage. We asked whether reprogramming of Foxp3+ Tregs to TH17-like cells also required RORγt. Rorγt^{gfp/gfp} have a knock-in of an EGFP sequence that disrupts the normal RORγt locus and homozygous-null mice are unable to upregulate IL-17 during TH17 differentiation. Tregs were isolated from Rorγt^{gfp/gfp} mice by sorting for CD4+CD25+ cells. FIG. 2B shows that Rorγt-null Tregs were unable to convert to IL-17 expression in our system. We next asked whether RORγt was required for the loss of functional Treg suppressor activity observed when IDO was blocked by D1MT (shown in FIG. 1C). FIG. 2C shows that wild-type Tregs activated in the presence of D1 MT lost all functional suppressor activity, as expected; whereas RORγt-deficient Tregs activated under identical conditions retained significant suppressor activity, even in the presence of D1MT. Thus, the loss of suppressor activity seen when IDO was blocked by D1MT represented an active, RORγt-dependent conversion of Tregs to the TH17 phenotype, not simply a passive failure of Treg activation.

IL-17 is expressed early during TH17 differentiation, whereas IL-22 is expressed later, and is thus is a marker of an established TH17 phenotype. FIG. 2D shows 4-color staining of Foxp3+ Tregs activated as above under (+)D1MT(+)OVA conditions. IL-22 was co-expressed by essentially all the Tregs that had upregulated IL-17. Thus, based on IL-17 expression, RORγt dependence, and co-expression of IL 22, the Tregs in our system converted to a phenotype similar to authentic TH17 cells.

Under strong proinflammatory conditions, CD4+ T-helper cells may co-express multiple cytokines (so-called polyfunctional T-helper cells) including IL-17, IL-2 and others. TH17 cells can co-express IL-2 in vivo, and TH17 cells differentiated in vitro can produce IL-2 and TNFα25. FIG. 2E shows that, in our system, most of the re programmed Tregs co-expressed IL-2 and TNFα, in addition to IL-17 and IL-22. Only a small number of reprogrammed cells expressed IFNγ or IL-10. Thus, reprogrammed Tregs appeared highly activated and a source of multiple proinflammatory cytokines.

### Example 3

### Upregulation of IL-17 in Tregs is Driven by IL-6

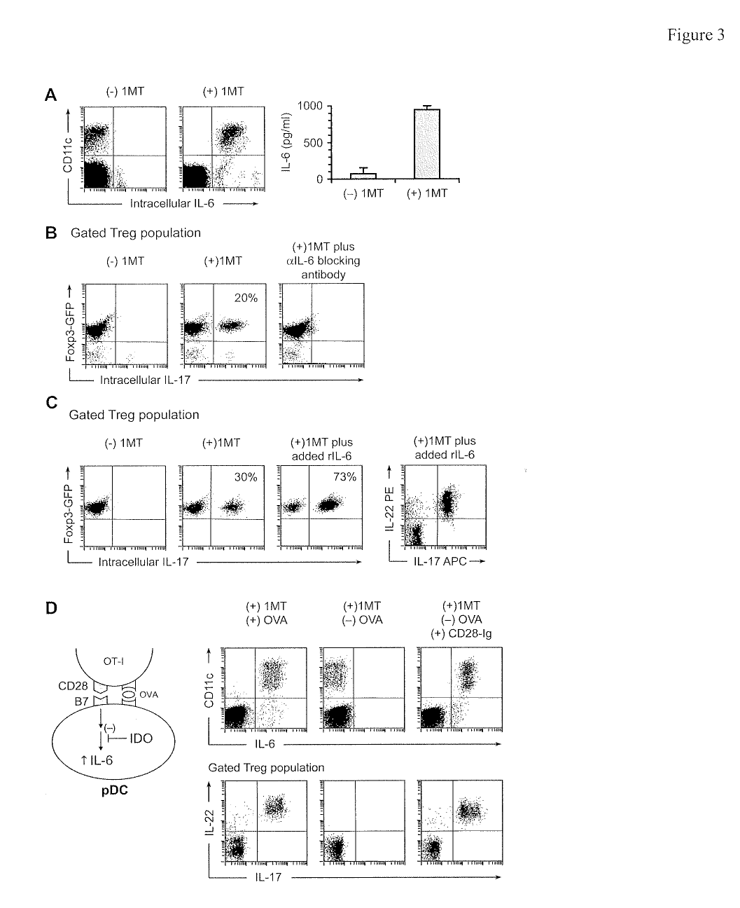

IL-6 is a proinflammatory cytokine that, in conjunction with TGFβ, can drive the differentiation of naive CD4+ T cells toward the TH17 lineage. Under certain conditions, IL-6 can be produced by activated pDCs, so we asked whether pDCs from TDLNs produced IL-6 in our co-cultures (FIG. 3A). For these studies, the feeder layers in co-cultures were depleted of macrophages (a potential contaminating source of IL-6). IL-17 upregulation in Tregs was unaffected by macrophage depletion, and essentially all of the IL-6-producing cells under these conditions were the pDCs (identified as CD11c+ in the FACS plots). IL-6 was expressed only when IDO was blocked with D1MT; if IDO was enzymatically active then IL-6 was suppressed. The suppressive effect of IDO was further confirmed by measuring IL-6 in co-culture supernatants by ELISA (FIG. 3A, right-hand panel)

To test whether IL-6 was mechanistically required for upregulation of IL-17 we used neutralizing anti-IL-6 antibody. FIG. 3B shows that blocking IL-6 completely abrogated upregulation of IL-17 in Tregs in co-cultures. Consistent with a mechanistic role for IL-6, addition of exogenous recombinant IL-6 to co-cultures drove even more conversion of Tregs, such that the large majority now became converted to the TH17-like phenotype (FIG. 3C).

### Example 4

### IL-6 Expression in pDCs is Triggered by Activated OT-I Cells

IL-6 induction in pDCs also required a signal from activated OT-I cells, in addition to IDO blockade. Thus, if OVA antigen was omitted from co-cultures, IL-6 was not induced even in the presence of D1MT (FIG. 3D, middle panel). This suggested that antigen presentation by pDCs to OT-I might be necessary to trigger IL-6 induction. During the process of antigen presentation, it is known that CD28 and its cognate B7 counter-ligands actively cluster in the immunologic synapse, and in other systems, CD28-mediated engagement of B7 can generate an intracellular “reverse” signal in DCs that triggers IL-6 production. Therefore, we asked whether the requirement OVA antigen in our system could be replaced by artificially cross-linking B7 molecules, using a recombinant CD28-Ig fusion protein. FIG. 3D, right panel, shows that CD28-Ig was able to fully substitute for the OVA signal, both for IL-6 induction in the pDCs, and for driving IL-17 and IL-22 upregulation in Tregs. These findings were thus consistent with a model (diagrammed in FIG. 3D) in which antigen-activated OT-I delivered an IL-6-inducing signal to pDCs via CD28-mediated engagement of B7 molecules.

### Example 5

### IDO Suppresses Expression of IL-6 in pDCs

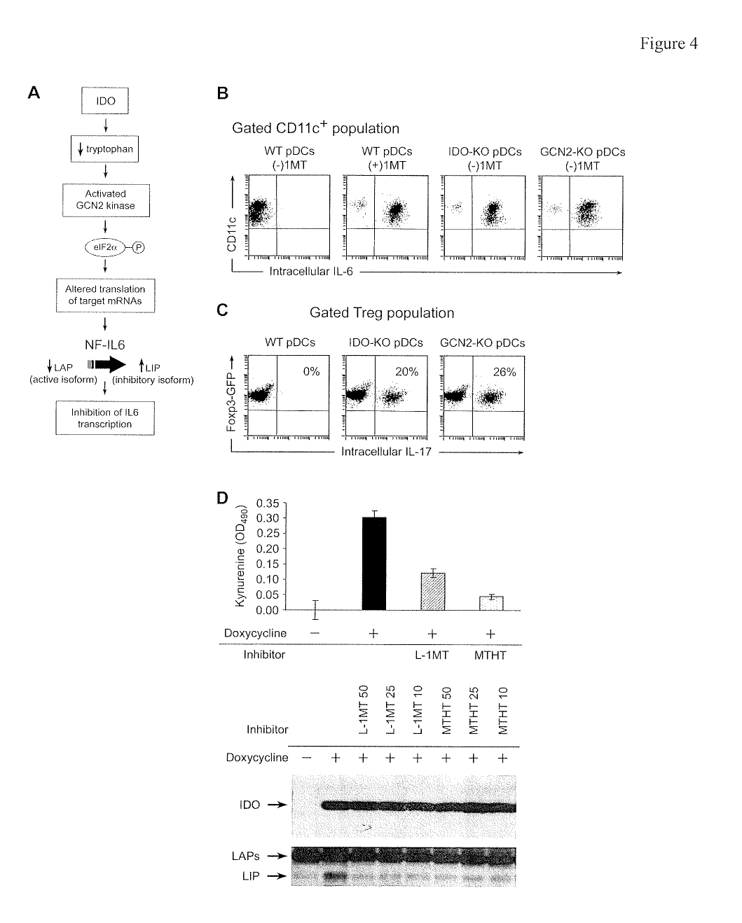

FIG. 3A had shown that IL-6 was produced by pDCs only when IDO was blocked by D1MT. This suggested that the IDO in pDCs might actively suppress their own production of IL-6. (We have previously demonstrated such an autocrine/paracrine effect of IDO on type I interferon production by pDCs.) IDO depletes the amino acid tryptophan, which can activate the amino-acid sensitive GCN2-kinase pathway, as diagrammed in FIG. 4A. Activated GCN2 phosphorylates ribosomal eIF2α, which alters translation of target mRNAs. One mRNA species known to be sensitive to amino-acid induced regulation is the transcription factor NF IL6 (C/EBPβ), which is a key regulator of IL-6 gene transcription Therefore, we asked whether IDO blocked IL-6 production by activating the GCN2 pathway in pDCs.

B16 tumors were grown in WT B6 mice, or in mice lacking IDO1 (IDO1 KO) or GCN2 (GCN2-KO). pDCs from TDLNs were used as APCs in activation co-cultures (the Tregs and other cells in co-culture were all from WT mice). FIG. 4B shows that pDCs lacking IDO1 were unable to suppress their own IL-6 production in co-cultures (i.e., even without D1MT, IL-6 was still expressed when pDCs lacked IDO1). Similarly, GCN2 KO pDCs we unable to suppress their own IL-6 production. Both IDO1 KO and GCN2 KO pDCs spontaneously drove conversion of Tregs to TH17-like cells in co-cultures, without the requirement for added D1MT (FIG. 4C).

The NF IL-6 (C/EBPβ) transcription factor exists in two forms: transcriptionally-active LAP (Liver-enriched transcriptional Activator Protein) isoforms that promote IL-6 transcription, and the dominant-negative LIP (Liver Inhibitory Protein) which inhibits LAP. Both LIP and LAP are generated from the same mRNA via alternate ribosomal start sites. GCN2 kinase is known to alter ribosomal initiation of many mRNA species. Therefore, we asked whether the IDO→GCN2 pathway might up-regulate the dominantly-inhibitory LIP isoforms of NF IL6. These mechanistic studies could not be performed on the small number of primary pDCs from TDLNs, so we used a model of T-REX cells transfected with a doxycycline-inducible IDO cDNA construct. FIG. 4D shows that induction of the IDO gene triggered up-regulation of the inhibitory LIP isoform of NF IL6, and that this was blocked by two different functional inhibitors of IDO enzymatic activity. Thus, taken together, our data are consistent with the hypothesis that IDO directly suppresses IL-6 induction, via GCN2-mediated regulation of NF IL6.

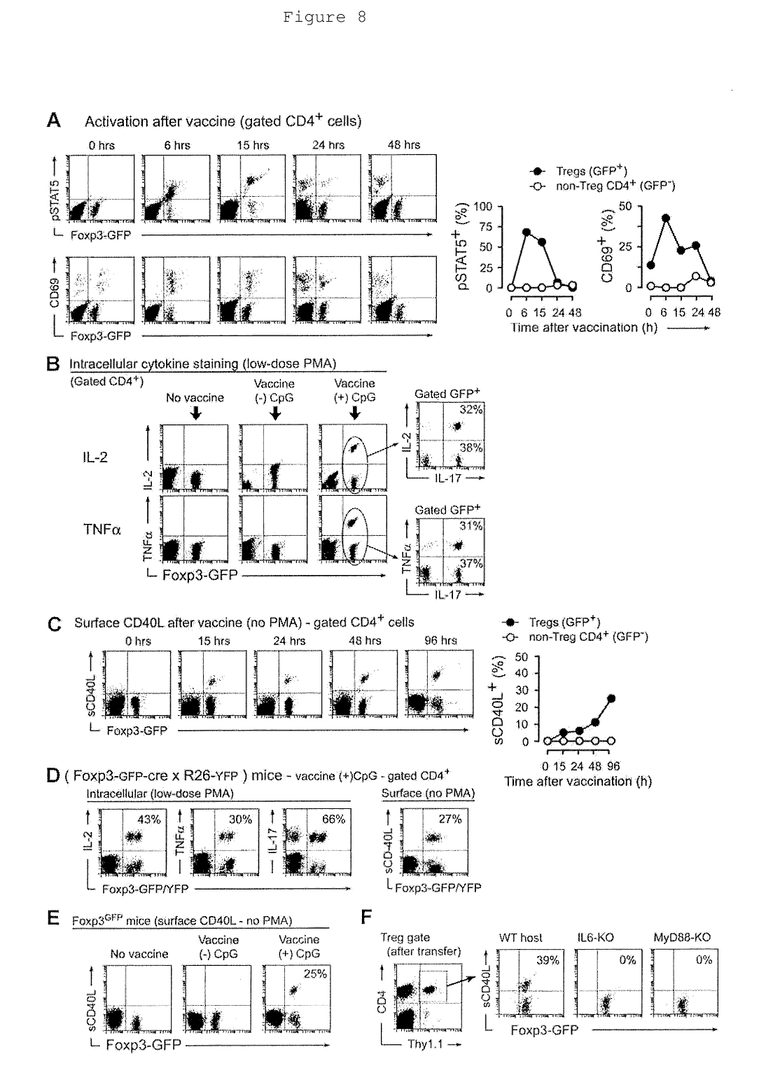

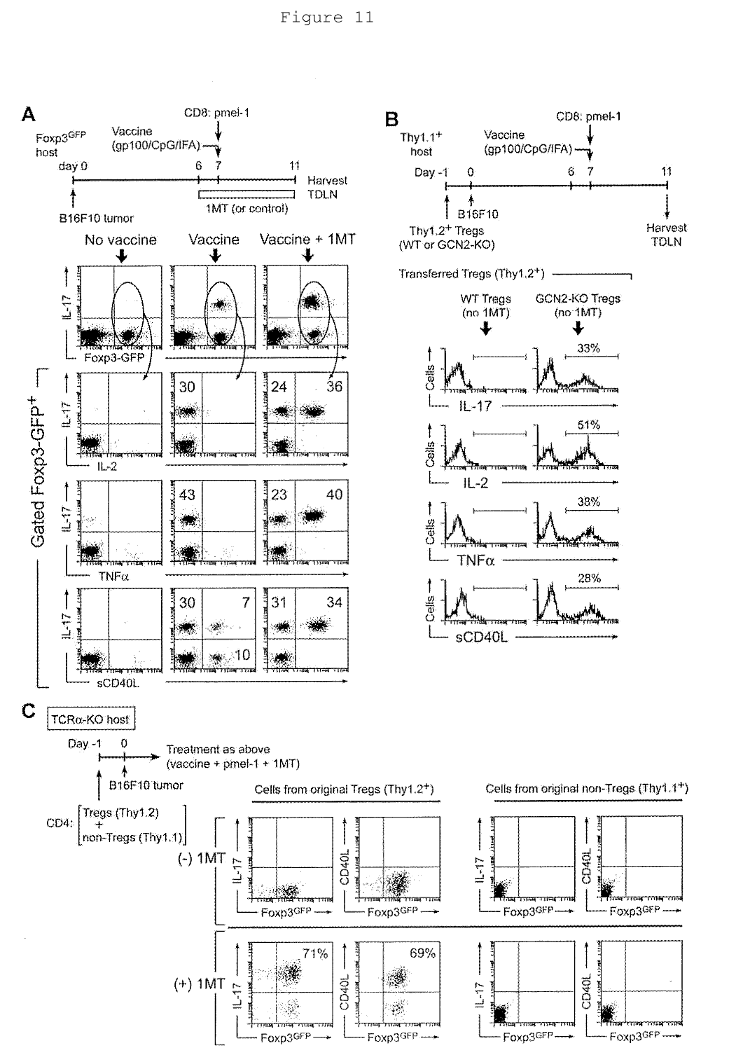

Mice lacking IL-6 did not support normal Treg reprogramming in response to vaccination (FIG. 8F). Additionally, IL-6 production by DCs can be directly suppressed by IDO in an autocrine/paracrine fashion via IDO-induced activation of the GCN2 pathways in the DCs (FIG. 4). IDO-induced inhibition of IL-6 thus provides one molecular pathway linking IDO expression in tumor bearing hosts with suppression of the normal Treg reprogramming pathway. In addition, we now demonstrate a second target for IDO, in which IDO acts directly on the Tregs themselves, via activation of their own GCN2-kinase pathway to inhibit reprogramming (FIG. 11). The importance of this Treg-intrinsic pathway was shown by the fact that Tregs lacking GCN2 were resistant to the effects of IDO, and remained able to reprogram even in tumor-bearing hosts. The GCN2 pathway is an important mediator of IDO-induced activation of Treg suppressor activity and GCN2 suppresses T117 differentiation in naïve CD4 T cells. Therefore, IDO acts both to suppress paracrine IL-6 and (via GCN2 in Tregs) to directly suppress Treg reprogramming.

### Example 6

### Replacement of Foxp3+ Tregs by TH17-Like Cells in Vivo

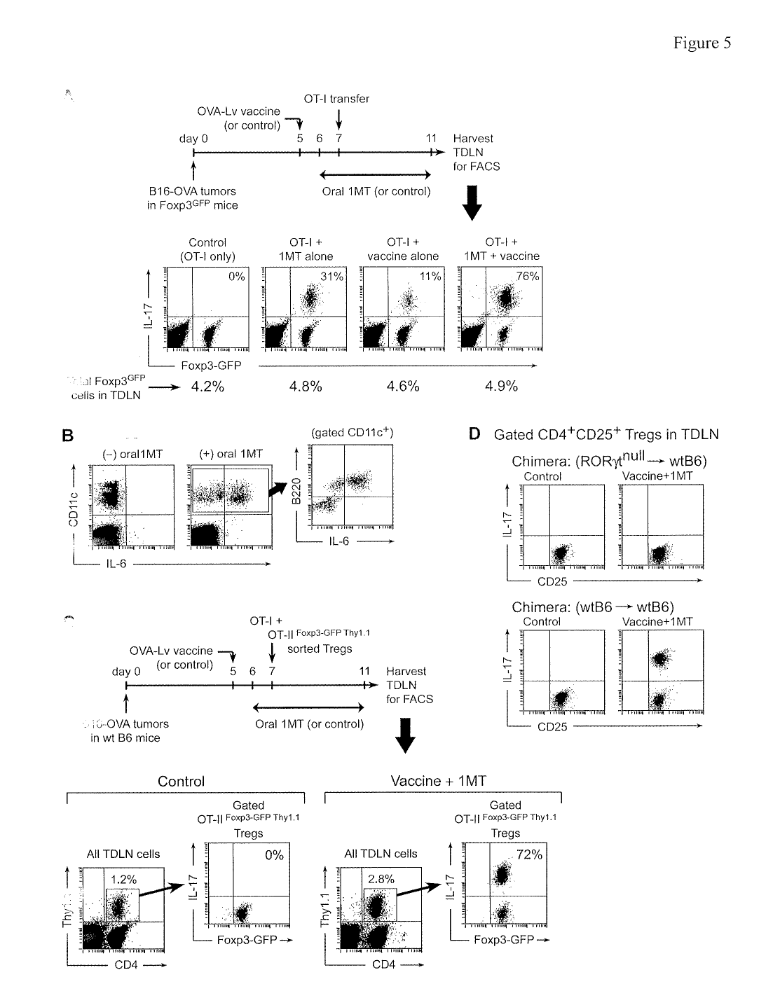

Our in vitro model showed that three cell types Tregs, pDCs and activated OT-I cells—needed to come together under conditions in which IDO was blocked in order to convert Tregs to TH17-like cells. This implies that a method of immunotherapy treatment based on reprogramming of Tregs into helper T cells must combine administration of an inhibitor of the IDO pathway and a vaccine. To test whether this interaction could occur in vivo, we used B16 tumor cells transfected with an ovalbumin transgene (B16-OVA) implanted in Foxp3^{GFP} mice. On day 7 of tumor growth, resting OT-I cells were adoptively transferred i.v., as shown in the schematic in FIG. 5A. Prior to adoptive transfer, mice were treated with or without oral D1MT in drinking water. To further drive activation of OT-I cells, some mice were immunized with a vaccine containing the OVA DNA sequence delivered in a lentiviral vector (OVA 1.v vaccine).

FIG. 5A shows that mice receiving only OT-I cells (control group) had no IL-17 expression by the endogenous Foxp3^{GFP} Tregs in TDLNs. Mice receiving OT-I plus concomitant D1MT administration showed a minority of Foxp3GFP cells converting to IL-17 expression (typically 25-30%). Mice receiving OT-I plus vaccine (without D1MT) showed little IL-17 expression. However, the combination of vaccine plus D1MT resulted in conversion of the majority of Tregs to IL-17 expression (up to >75% co expression of Foxp3^{GFP} and IL-17, as shown in the fourth panel in FIG. 5A). In all groups, the total percentage of Foxp3^{GFP} expressing cells in the TDLNs remained constant (shown as the percentages below each dot-plot in FIG. 5A), with the change occurring in the relative fraction of cells co-expressing IL-17.

Further consistent with the predictions of our in vitro model, many of the pDCs in TDLNs upregulated IL-6 when challenged with OT-I cells in the presence of D1MT (FIG. 5B). Typically 2-3% of total TDLN cells were found to be DCs (defined as CD11c+); within these, the expression of IL-6 was confined to the CD11c+B220+ (plasmacytoid DC) fraction, as shown in the gated population in FIG. 5B. (In these studies, the LN disaggregation protocol was optimized for recovery of pDCs, so recovery of myeloid DCs may not have been quantitative; but qualitatively the expression of IL-6 was confined to the pDCs.)

### Example 7

### Direct Conversion of Mature Foxp3+ Tregs to TH17-Like Cells in Vivo

In FIG. 5A, the presence of residual Foxp3^{GFP} fluorescence in essentially all of the IL-17-expressing cells suggested that the IL-17+ cells might arise from conversion of pre existing Foxp3+ Tregs (which we had shown to occur in our in vitro model). To test this, wild-type B6 mice with B16 OVA tumors were immunized in the presence of D1MT, and a defined population of mature, Foxp3+ Tregs were adoptively transferred at the time of OT-I injection (FIG. 5C). The transferred Tregs were isolated from TCR-tg OT-II mice (CD4+, specific for a peptide of ovalbumin) that had been crossed with Foxp3^{GFP} mice, and bred on a Thy1.1 congenic background (described in Example 1). OT-II^{Foxp3 GFP Thy}1.1 Tregs were sorted as CD4+GFP+ cells, and thus were known to be uniformly Foxp3+ at the time of transfer. FIG. 5C shows that in control recipients (without vaccination or D1MT), none of the transferred OT-II^{Foxp3 GFP Thy}1.1 Tregs in TDLNs converted to IL-17 expression. However, in mice treated with OVA Lv vaccine and D1MT, the majority of transferred Tregs in TDLNs upregulated IL-17. These IL-17-expressing cells were unambiguously identified as the transferred Tregs by the Thy1.1 congenic marker, and retained residual Foxp3^{GFP} fluorescence (just as in our in vitro model). Thus, these studies formally demonstrated that mature pre-existing Foxp3+ Tregs could be directly converted to the IL-17-expressing phenotype in vivo. For the studies shown, we chose OT-II Tregs with a TCR recognizing a tumor antigen, as used by others (Wang, et al., 2008; Proc. Natl. Acad. Sci., USA, 105:9331-9336), but we obtained similar results using polyclonal natural Tregs from Foxp3^{GFP} donors, so the observed in vivo reprogramming was not restricted to OT-II cells.

FIG. 5D shows that upregulation of IL-17 by Tregs in TDLNs also required an intact RORγt transcription factor in the Tregs (consistent with the in vitro model shown in FIG. 2B). For these studies, the tumor-bearing hosts were bone-marrow chimeras of RORγtnull marrow transplanted into wt B6 hosts, since the RORγt-deficient mice themselves are defective in peripheral LN development.

### Example 8

### Enhanced Anti-Tumor Response to Vaccine Plus D1Mt

We next asked whether replacement of Tregs by TH17-like cells in TDLNs was associated with enhanced functional anti-tumor immune response. We first addressed this question in the nominal B16-OVA system, where the CD8+ effector cells were known. B16-OVA tumors grow aggressively in immunocompetent hosts, despite the potent xenoantigen; and once established, tumors induce unresponsiveness in naive OT-I cells and convert naive CD4+ OT-II cells into adaptive Tregs. Thus, B16 OVA is informative because the artificial antigen is highly immunogenic, yet the anti-tumor immune response is suppressed.

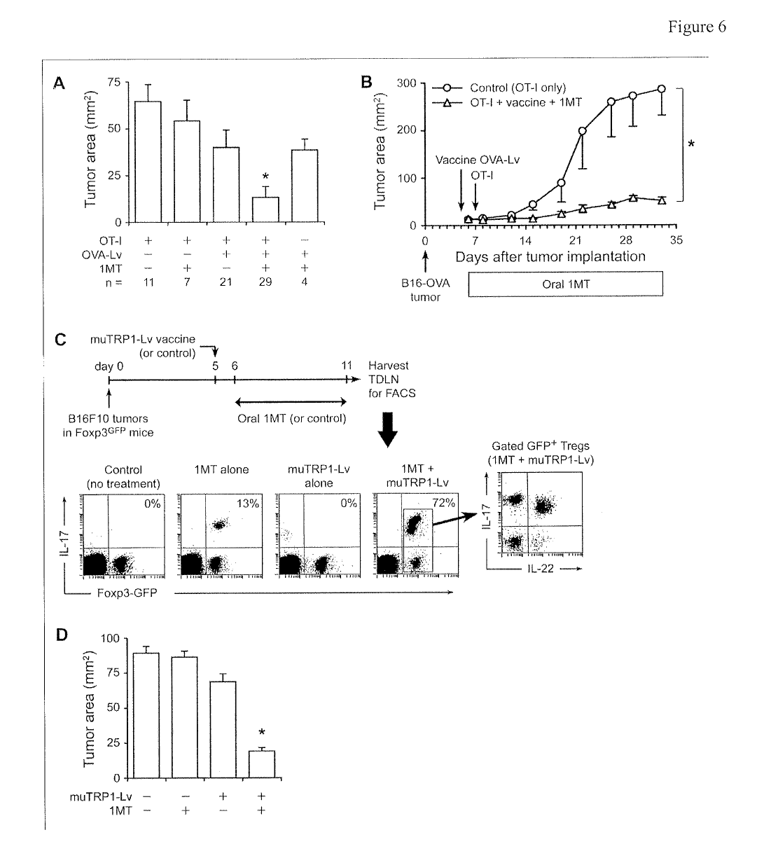

Mice with B16-OVA tumors received various combinations of OVA Lv vaccine, D1MT in drinking water, and OT-I adoptive transfer as indicated in FIG. 6A (delivered via the same protocol as in FIG. 5A). On day 11, tumors were measured in situ at necropsy. (Day 11 was chosen because even partial responses were evident at this time point; whereas at later time-points minor differences became obscured as tumors grew out. The maximum reduction in tumor size on day 11 was obtained by adding D1MT to the regimen of vaccination+OT-I, corresponding to the conditions which produced maximum conversion of Tregs to TH17-like cells (cf. FIG. 5A). When followed for a longer period, tumors treated with D1MT plus OT-I and vaccine showed sustained growth delay (FIG. 6B).

### Example 9

### Treg Conversion can be Driven by Endogenous T Cells Against a Shared Self/Tumor Antigen

The OVA system was informative for mechanistic studies, but a more realistic clinical scenario is vaccination against a shared self/tumor antigen to which the host is already tolerant. Under these conditions, it was not clear whether there would be adequate endogenous CD8+ T cell response to drive conversion of Tregs to TH17-like cells. To test this, we used an altered peptide ligand sequence developed against the melanoma-associated antigen P1, optimized to break tolerance to native TRP 1 in tumor-bearing hosts. The muTRP-Lv vaccine was delivered via the same recombinant lentivirus vector used above to deliver OVA36. B16F10 tumors were grown in Foxp3^{GFP} knock-in mice, and mice were immunized with muTRP1 Lv vaccine, with or without oral D1MT, as shown in FIG. 6C. Mice receiving D1MT alone showed few GFP+Tregs converting to IL-17 expression, and mice receiving vaccine alone showed minimal conversion. However, mice receiving the combination of muTRP1 Lv vaccine and D1MT showed conversion of a large majority of Tregs in TD1,Ns into TH17-like cells. Thus, vaccination against an endogenous shared self/tumor antigen was able to drive extensive reprogramming of Tregs when combined with D1MT.

Similar to the nominal OVA system, reprogramming of Tregs was associated with enhanced functional anti-tumor responses to muTRP1-Lv vaccine, measured by tumor size on day 11 (FIG. 6D). As in the B16 OVA experiments, a large inoculum of B16F10 tumor cells (1×10⁶) and an early time-point were used; under these stringent conditions, vaccine and D1MT were each minimally effective as single agents, but the combination of vaccine+D1MT showed significant synergistic anti-tumor effect.

### Example 10

### D1MT Enhances Response to CpG-Based Vaccine

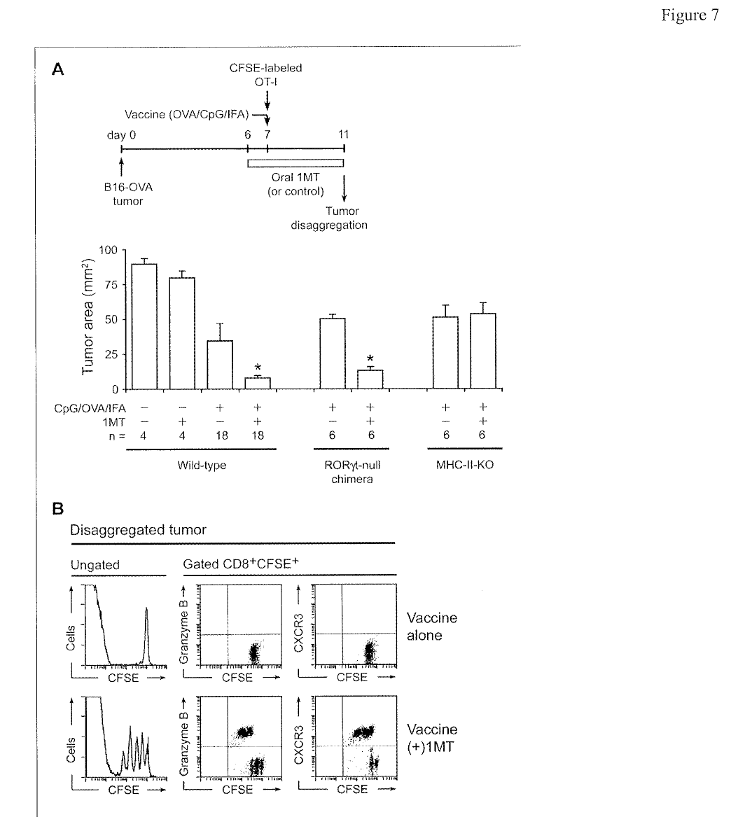

To confirm that the effect of D1MT was not restricted only to lentivector vaccines, we tested D1MT with a vaccine comprising OVA protein emulsified in incomplete Freund's adjuvant plus CpG oligodeoxynucleotide 1826, a TLR9 ligand. FIG. 7A shows that this vaccine by itself had only modest effect against established (day 7) B16 OVA tumors, but that the addition of D1MT showed significant synergy with vaccine. When similar studies were performed in RORγt-null bone-marrow chimeric mice (RORγt-null marrow→B16 hosts, as in FIG. 5D), the synergistic effect of D1MT was preserved, indicating that the RORγt/IL-17 pathway itself was not indispensable for anti-tumor effect of D1MT (FIG. 7A). However, we noted that the RORγt pathway is selective for IL-17, and RORγt-null Tregs could still upregulate IL-22 and undergo other pro-inflammatory changes. Therefore, we asked whether mice lacking all CD4+ T helper cells (not just RORγt/IL-17) were still able to respond to vaecine+D1MT. For these studies we used MHC class ft-deficient mice (IA^{b}-KO mice), which lack all detectable CD4+ T cells (both Tregs and T-helper cells). In these mice, the synergistic effect of D1MT was completely abrogated (FIG. 7A). Thus, the helper activity of CD4 cells appeared required for the synergistic effect of D1MT.

In the tumors themselves, CFSE-labeled OT-I cells showed better ability to divide and upregulate differentiation markers (granzyme B and CXCR3) in mice treated with D1MT+ vaccine, compared to vaccine alone (FIG. 7B). Indeed, proliferation of OT-I in these large established tumors was poor in the absence of D1MT, reminiscent of the reported suppression of OT-I by other established tumors. In these studies, as with the lentivector experiments above, stringent conditions (large established tumors) were chosen to favor suppression.

According to the current invention, the phenotype of re-programmed Tregs was similar to activated TH17 cells or to “polyfunctional” T-helper cells, since they co-expressed both IL-17 and IL 22 (associated with the TH17 lineage), and also IL-2 and TNFα. Some TH17 cells are known to co-express other cytokines, such as IL-2. According to the current invention, we refer to the re programmed Tregs as “TH17-like” because of their RORγt-dependent induction of IL-17 expression; but whether they are considered TH17 cells or polyfunctional T-helper cells is largely a matter of semantics. The important mechanistic finding of the current invention is that they are a potent source of helper cytokines. Our studies with CD4-deficient mice (MI-IC-II-KO) suggests that CD4+ T-helper cells play an indispensable role in the synergistic anti-tumor effects of D1MT. These helper effects are more than just the proinflammatory effects of IL-17, as shown by the studies with RORγt-null mice. According to the current invention, the helper cytokines from re programmed Tregs are an important mechanism of CD4+ help in vivo in the setting of vaccination plus IDO-blockade.

### Example 10

### Tregs Undergo Reprogramming in Vaccine-Draining Lymph Nodes

Treg reprogramming was studied using a vaccination model in which a protein antigen (whole chick albumin, OVA) must be processed by DCs and cross presented on MHC class I to CD8⁺ T cells. In this model, CD8⁺ T cell activation is dependent on CD4⁺ help to license the DCs for successful cross presentation. Vaccine recipients were C57BL/6 mice bearing a FoxP3-GFP fusion protein at the FoxP3 locus, which marks the FoxP3′ Tregs with high fidelity. We have previously shown that Tregs continue to display detectable GFP fluorescence for at least 4 days after reprogramming, allowing us to follow the Tregs during and after conversion. FoxP3^{GFP} mice received adoptive transfer of a defined responder cohort of OVA-specific OT-I (CD8⁺, recognizing the SIINFEKL peptide of OVA), followed by immunization with whole OVA protein plus the TLR9-ligand CpG-1826, emulsified in IFA.

FIG. 8A shows analysis of CD4⁺ cells in the draining lymph node (LN) during 6-48 hours following immunization. Tregs and conventional (non-Treg) CD4⁺ cells are distinguished based on FoxP3-GFP expression. Previous reports suggested that Tregs respond rapidly to certain proinflammatory signals; based on this we tested phosphorylation of STAT5 (an activation pathway downstream of the IL-2 and other γe cytokine receptors). Large numbers of GFP⁺ Tregs did not respond until 24-48 hours after immunization. Similar results were seen with the early activation marker CD69 (lower panels). Thus in this model, Tregs were the first to respond to vaccine-induced activation.

We next asked whether activated Tregs showed evidence of phenotypic plasticity (reprogramming) following vaccination. One defining characteristic of plasticity is that Tregs acquire the ability to express proinflammatory cytokines such as IL-2, IL-17 or TNFα when stimulated in vitro with agents such as PMA/ionomycin. This inducible cytokine expression implies a major alteration in the underlying Treg phenotype, because normally these pro inflammatory genes would be profoundly suppressed in the Foxp3+ lineage. The standard protocol to detect inducible cytokine production relies on activation with PMA/ionomycin in the presence of an inhibitor of protein export, followed by intracellular cytokine staining. In preliminary studies, we found that the usual PMA/ionomycin reagents caused artifactual activation of large numbers of resting CD4+ T cells, which obscured the specific effect of vaccination. This nonspecific background could be minimized by using a 10-fold lower concentration of PMA/ionomycin, under which conditions the high-responsive Tregs continued to respond robustly. FIG. 8B shows that prior to vaccination, resting Tregs from Foxp3^{GFP} mice produced no IL-2 or TNFα when challenged with PMA, as expected (left-hand panels). However, after vaccination (right-hand panels) many Tregs had acquired the ability to produce IL-2 and TNFα, and large numbers also co-expressed IL-17, suggesting acquisition of a polyfunctional “helper-like” phenotype. Treg reprogramming required the inflammatory signal provided by the CpG adjuvant, because most cytokine production was lost if CpG was omitted from the vaccine (middle panels).

Inducible cytokine production was an informative readout, but it required in vitro manipulation. We therefore examined the in vivo induction of cell-surface CD40-ligand (CD40L), an important functional mediator of T cell help. CD40L was measured directly ex vivo, without any stimulation. FIG. 8C shows that CD40L was upregulated on a subset of Tregs beginning at approximately 15 hrs after vaccination. Somewhat fewer Tregs expressed constitutive CD40L than could be induced to express cytokines with PMA, but up to 25% of all Tregs constitutively upregulated surface CD40L. Additional studies, not shown, confirmed that CD40L was expressed on the population of reprogrammed Tregs that also co-expressed multiple pro inflammatory cytokines (polyfunctional phenotype). Thus, while Tregs are known to express inducible or intracellular CD40L under various conditions, in our model constitutive surface CD40L, appeared an informative marker of the reprogrammed phenotype. Of note, FIG. 8C also shows that, during this early phase of a priming immunization under our experimental conditions, the only cells expressing CD40L were derived exclusively from the Treg population (GFP+), and there was no expression by conventional CD4+ T cells. Importantly, the current invention demonstrates that the conversion of Tregs to helper T cells was remarkably and surprisingly rapid. Tregs in vaccine-draining LNs began to express cell surface CD40L within 15 hrs of vaccination, whereas conventional CD4 cells showed no constitutive CD401, expression even after 4 days. This rapid and widespread conversion, coupled with the non-redundant functional role of Tregs as helper cells in our vaccination model, suggests that the natural biological role of FoxP3 lineage cells includes both helper and suppressor functions, depending on the cues from the local microenvironment.

Results from the Foxp3^{GFP} knock-in mice were confirmed in a second reporter strain, hearing a Bac-transgenic MT/ere recombinase construct under the Foxp3 promoter. When this mouse is crossed with a ROSA26R-YFP (floxed-stop) reporter strain, the offspring have >95% of Foxp3+ cells irreversibly marked by YFP. FIG. 81) shows that immunization of these mice (using the same OT-I adoptive transfer and OVA/CpG vaccine protocol as in FIG. 8A) likewise caused phenotypic alteration revealed by inducible cytokine expression, and also constitutive upregulation of CD40L expression; and again this was confined exclusively to the Foxp3^{GFP/YFP} Treg population. (For this analysis, GFP and YFP cells are combined in the FL1 channel, to capture all Foxp3-lineage cells.)

FIG. 8E shows that the acquisition of constitutive CD40L, expression required the presence of CpG in the vaccine (just as we found for the acquisition of inducible cytokine production in FIG. 8B, above). In previous reports, CpG has been shown to block the suppressor activity of Tregs in vivo via a pathway requiring MyD88, the signaling adapter downstream of TLR9. Consistent with this, FIG. 8F shows that Tregs adoptively transferred into hosts lacking MyD88 became unable to upregulate CD40L on Tregs in response to CpG vaccine (likewise, induction of pro inflammatory cytokines was also abrogated or markedly reduced in MyD88 KO hosts, data not shown). IL-6 is a key pro inflammatory cytokine that drives Treg reprogramming in vitro. In our model, Tregs transferred into hosts lacking IL-6 became unable to upregulate CD40L in response to vaccination (FIG. 8F), and had absent or markedly reduced upregulation of pro inflammatory cytokines (not shown). Thus, consistent with the effect of IL-6 in vitro models, host IL-6 appeared to be a key driver of Treg reprogramming in vivo, and was part of the innate inflammatory milieu created by TLR9 ligation in an MyD88-dependent fashion.

### Example 11

### Reprogrammed Tregs are Required for CD8 Responses to Cross-Presented Vaccine Antigen

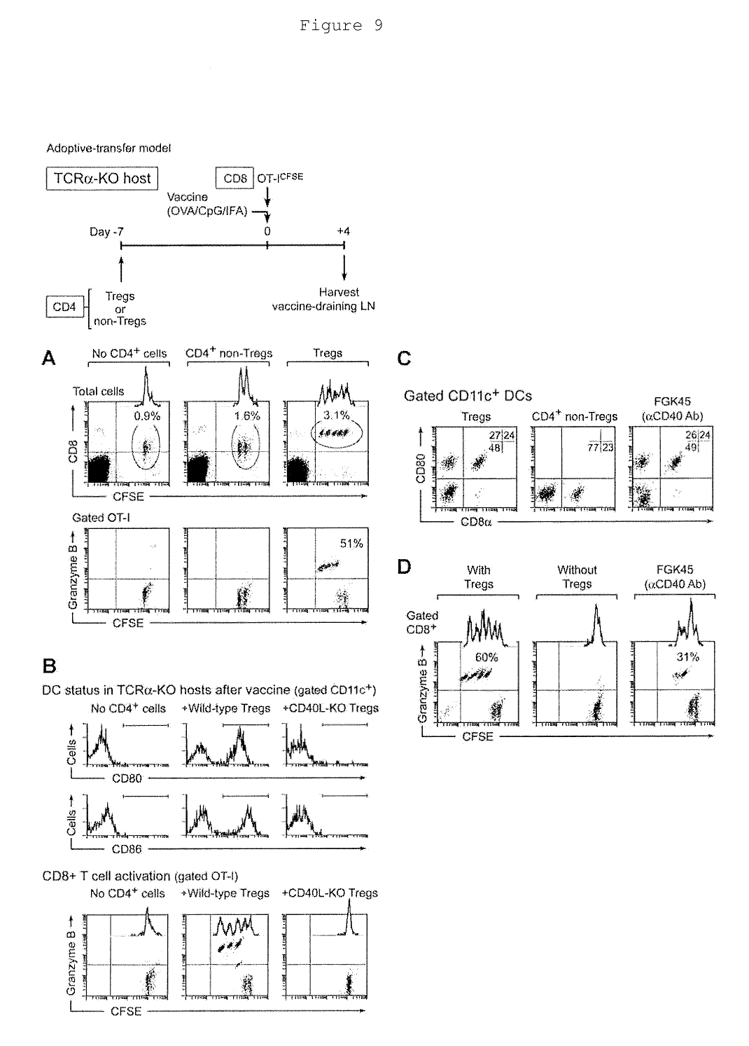

To test whether the reprogrammed Tregs could provide functional helper activity for CD8 cells, we selectively reconstituted TCRα-chain knockout (TCRα KO) mice with defined populations of CD4+ cells. TCRα KO mice have B cells, NK cells and γδ T cells, and so are not globally lymphopenic, but selectively lack αβ T cells (and thus have no endogenous CD4+ or CD8+ T cells). TCRα KO hosts received either sorted Foxp3^{GFP} Tregs, sorted non-Tregs (CD4⁺GFP^{NEG} cells from the same mice), or no CD4+ cells. Mice were rested, then received CFSE-labeled OT-I cells and were immunized with OVA/CpG/IFA vaccine, as shown in the diagram in FIG. 9. If TCRα KO mice received no CD4+ cells (FIG. 9A, left-hand panels), then OT-I cells showed little proliferation, poor cell recovery (few CD8+ OT-I cells in vaccine-draining LN), and no upregulation of the differentiation marker granzyme B. Adoptive-transfer of conventional (non-Treg) CD4+ cells also failed to provide effective help for OT-1 cells (middle panels), allowing at most a single round of cell division and no phenotypic maturation (granzyme B expression). In contrast, adoptive-transfer of the Treg fraction provided effective help, driving robust OT-I proliferation and granzyme B upregulation (right-hand panels). Since together the GFP+ and GFP^{NEG} fractions comprised all of the CD4+ T cells in the donor mice, our results revealed that essentially all of the available helper activity in our model was being contributed by the Treg fraction, not by the conventional CD4+ cells.

This potent helper activity within the Foxp3+ Tregs lineage was not an artifact of Tregs derived from the Foxp3^{GFP} knock-in strain, because sorted CD4+CD25+Tregs from wild-type B6 mice produced identical results, and sorted Tregs from Foxp3-GFP-cre×ROSA26R-YFP donors (as used in FIG. 8D) were also excellent helper cells in the TCRα KO model (data not shown).

### Example 12

### CD40L on Reprogrammed Tregs Drives DC Activation and CD8+ T Cell Proliferation

CD40L is a key molecular mechanism allowing helper T cells to activate (“license”) DCs so that they can cross-present antigens to CD8+ T cells. We asked whether CD40L expression on converted Tregs allowed them to activate host DCs. As an indicator of DC activation, we used expression of the costimulatory molecules CD80 and CD86. TCRα KO mice received adoptive transfer of Tregs from WT donors or from CD40L KO donors (controls received Tregs); then all mice received OT-1 and OVA/CpG/IFA vaccine, as in the previous experiment. FIG. 9B (left-hand panels) shows that in TCRα KO mice receiving no Tregs, the DCs in vaccine-draining LNs were unable to upregulate CD80 and CD86; and the paired CFSE histogram below shows that OT-I cells did not divide or express granzyme B. In hosts receiving WT Tregs (middle panels), CD80 and CD86 were highly upregulated on DCs after vaccination, and OT-I divided robustly and upregulated granzyme B. In contrast, mice reconstituted with Tregs that lacked CD40L (right-hand panels) showed no upregulation of CD80 and CD86 on DCs, and no proliferation of OT-I.

The CD8α+ subset of DCs is the population primarily responsible for cross-presentation to CD8+ cells. Using the same TCRα KO/vaccination model. FIG. 9C (left plot) shows that adoptive transfer of Tregs drove upregulation of costimulatory molecules on essentially all of the CD8α DCs, and on some of the CD8α^{NEG} DCs as well (CD80 is shown, similar results were seen for CD86). In contrast, adoptive transfer of the non-Treg CD4+ population (middle plot) failed to support activation of any DCs in TCRα KO mice after vaccination (this was consistent with the lack of helper activity by these non-Treg CD4+ cells in FIG. 9A). The effect of Tregs could be largely replaced by injecting mice with an activating antibody against CD40, which mimics the effect of CD40L. This antibody also bypassed the functional requirement for Tregs to support OT-I activation (FIG. 9D). Thus, taken together, our data indicated that reprogrammed Tregs delivered help to CD8+ T cells in large part via CD40L; and that it was the Treg lineage, not conventional CD4+ T cells, that was the source of CD40L-mediated help. This requirement for CD40L-mediated help to support CpG-based vaccination was consistent with earlier reports showing that TLR-driven inflammation, by itself, has minimal effect on priming CD8+ responses unless combined with activation of the CD40/CD40L pathway.

### Example 13

### Conventional Non-Treg CD4⁺ Cells can Deliver Help if they are Antigen-Specific

The lack of helper activity in the conventional CD4+ cells was unexpected, because these are the classical “T-helper” cells. However, our model featured a priming immunization in naive mice, and the naive CD4+ repertoire contains an extremely low frequency of clones specific for any given antigen. We hypothesized that the failure of conventional CD4+ cells to provide help was due to the low number of OVA-specific clones. (In contrast, Tregs, with their high frequency of self-reactive TCRs, would not be subject to this limitation.) To determine whether higher clonal frequency would allow cognate help from conventional (non-Treg) CD4+ cells, we used OT-II mice, which have TCR-transgenic CD4+ cells specific for an epitope of OVA. In OT-II transgenic mice the non-Treg fraction did indeed provide help for OT-I cells, whereas the same non-Treg fraction from non-transgenic (WT) mice did not. Thus, the unique feature of Tregs was not that they were more responsive than conventional T helper cells, but rather that they could spontaneously provide help even in naive (unprimed, non-transgenic) mice, when sufficient antigen-specific conventional CD4+ cells were not yet available. Therefore, vaccination in a way that induces Treg cell reprogramming (in conditions of effector T cell stimulation and simultaneous blockage of the IDO pathway) can be more effective than classic vaccination approaches.

### Example 14

### Established B16 Tumors Induce Progressive Unresponsiveness to Vaccine

We next evaluated the response to vaccination in mice with established tumors. This is a very different setting from vaccination of naive mice, because established tumors actively inhibit T cell responses against tumor-associated antigens. We used the B16 melanoma model (clone B16F10) because this tumor potently suppresses CD8 responses, and once tumors become established (day 4-5) anti-tumor vaccines become essentially ineffectual.

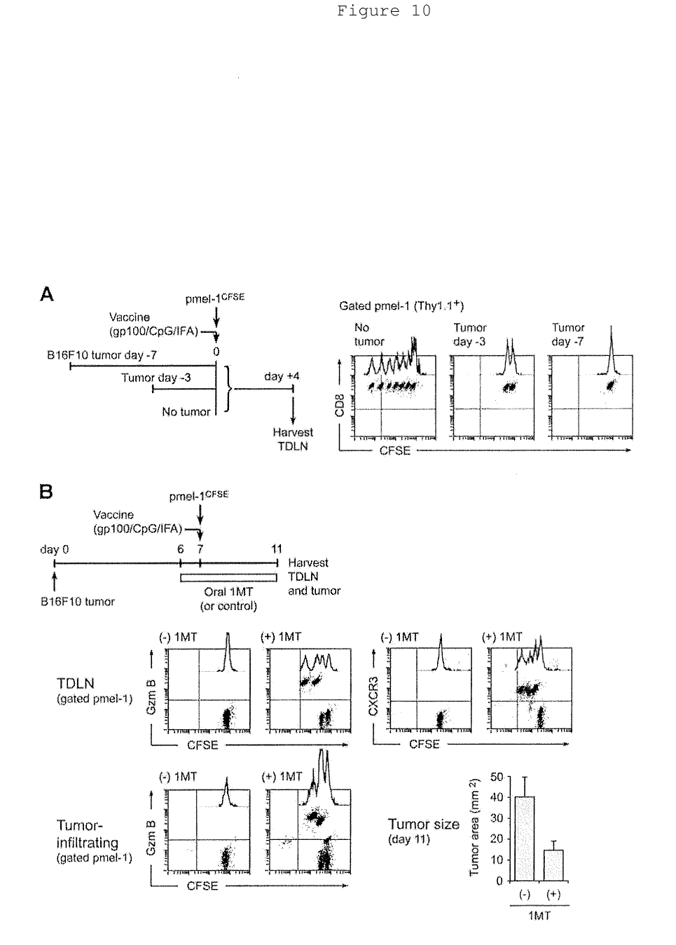

FIG. 10 demonstrates the progressive loss of T cell responsiveness to vaccination during B16F10 tumor growth. Mice with tumors of different durations received adoptive transfer of TCR-transgenic CFSE-labeled pmel 1 cells (CD8+, recognizing the shared self/tumor antigen gp100). Mice were then vaccinated with an altered peptide ligand vaccine (human gp100) emulsified in CpG/IFA, and proliferation of pmel 1 measured 4 days later. In mice without tumors, this vaccine produced robust proliferation of pmel 1 (first panel). However, in mice with day 3 tumors, response to vaccine was markedly reduced (second panel), and by day 7 of tumor growth the response to vaccination was essentially lost (third panel). The kinetics of suppression depended on the tumor cell line and the size of the inoculum (we used a large inoculum of 1×10⁶ B16F10 cells), but similar loss of responsiveness was seen with E.G7 lymphoma tumors (data not shown), and this is consistent with reports by others.

### Example 15

### Tumor-Induced IDO Blocks Treg Reprogramming

We have previously shown that 1316 tumors upregulate host expression of the immunosuppressive enzyme IDO. In vitro, IDO can stabilize the suppressive phenotype of Foxp3⁺ Tregs, and antagonize their reprogramming into helper-like cells. Therefore, we asked whether IDO in tumor-bearing hosts suppressed Treg reprogramming in vivo. FIG. 11A (middle set of dot-plots) shows that mice with established tumors displayed impaired reprogramming of Tregs in tumor-draining LN following vaccination (reduced IL-17 and CD40L expression, and complete loss of IL2 and TNFα expression). In contrast, when IDO was blocked with the IDO-inhibitor drug 1-methyl-D-tryptophan (D1MT), then the same vaccination cause extensive Treg reprogramming (right-hand dot-plots). (The figure shows data from tumor-draining LNs; similar results were also seen in LNs draining the vaccination site.)

To confirm that IDO was acting directly on Tregs, we asked whether Tregs that lacked the GCN2-kinase pathway would be resistant to the effects of IDO (i.e. would undergo normal reprogramming even when IDO was active). GCN2 senses amino-acid deprivation, and we have shown that this signaling pathway is required in target T cells in order for IDO to exert many of its regulatory effects. FIG. 11B shows experiments in which a cohort of congenic Tregs (Thy1.2⁺) was enriched from either GCN2-KO mice or WT B6 controls and transferred into Thy1.1⁺ hosts. Mice then received tumors and were vaccinated 7 days later, but were not treated with D1MT. Under these conditions (IDO active), there was little detectable reprogramming of the WT Treg cohort, as expected; however, the Treg cohort from GCN2-KO mice were resistant to the effects of IDO, and underwent normal reprogramming following vaccination, despite the presence of tumor, and without the need for D

Taken together these data indicates that established tumors progressively create a milieu in tumor-bearing hosts in which the normal, vaccine-induced reprogramming of Treg cells was suppressed and could no longer occur. This inhibition of reprogramming was mediated by tumor-induced IDO and could be reversed by pharmacologic inhibition of the IDO pathway in concurrence with vaccination.

### Example 16

### Vaccine-Activated Helper Cells Derive Preferentially from Reprogrammed Tregs when EDO is Blocked

It was formally possible that the cytokine-expressing GFP+ cells seen in FIG. 11A (which we termed reprogrammed Tregs), might actually arise from naive CD4+ cells that had upregulated Foxp3 de novo (along with the cytokines). To definitively establish the origin of these activated, cytokine-expressing, CD40L+ cells, we used TCRα-KO hosts reconstituted with congenially-marked subsets of CD4+ cells. The Treg subset was sorted from Foxp3^{GFP} donors (Thy1.2+) and the non-Treg subset (CD4+GFP^{NEG}) was sorted from congenic Foxp3^{GFP-Thy}1.1 donors. Cells were then mixed in the original ratio (1:5) and transferred into TCRα-KO hosts. The reconstituted hosts were then inoculated with B16F10 tumors, and 7 days later received pmel-1 cells and gp100 vaccination, with or without D1MT. FIG. 11C shows that after vaccination, all of the CD4+ cells that expressed pro-inflammatory cytokines and CD40L, derived exclusively from the original Treg population; whereas the non-Treg population remained quiescent and contributed none of these activated helper cells.

### Example 17

### Reprogrammed Tregs Are Required to Support CD8⁺ Response to Anti-Tumor Vaccine

We next asked whether blocking IDO could restore the anti-tumor CD8⁺ responses that were lost in hosts with established tumors (cf. FIG. 10). FIG. 121 shows that responses of pmel-1 in tumor-bearing hosts could be restored if IDO was blocked by treating mice with D1MT at the time of vaccination. Without D1MT, pmel-1 cells were suppressed in draining LNs of day 7 established tumors, but in mice treated with D1MT, pmel-1 cells became able to divide and upregulate granzyme B and the chemokine receptor CXCR3 (a functionally important marker of CD8 maturation, because it is required for homing to sites of inflammation. Within the tumor itself, activated, proliferating pmel-1 cells were found in D1MT-treated mice (but not in mice without D1MT). As a proxy for functional anti-tumor effect, tumors were dissected at the end of the experiment (day 11) and the size measured, as shown in the bar graph. This was not a measure of long-term tumor regression, because all tumors eventually re-grew following a single dose of vaccine; however, the rapid and significant reduction in tumor size compared to controls served as useful experimental confirmation that the proliferating, granzyme B-expressing pmel-1 cells were associated with biologically-relevant anti-tumor activity.

In other confirmatory experiments (data not shown) similar effects of D1 MT on vaccination was seen in the E.G7 tumor system as well.

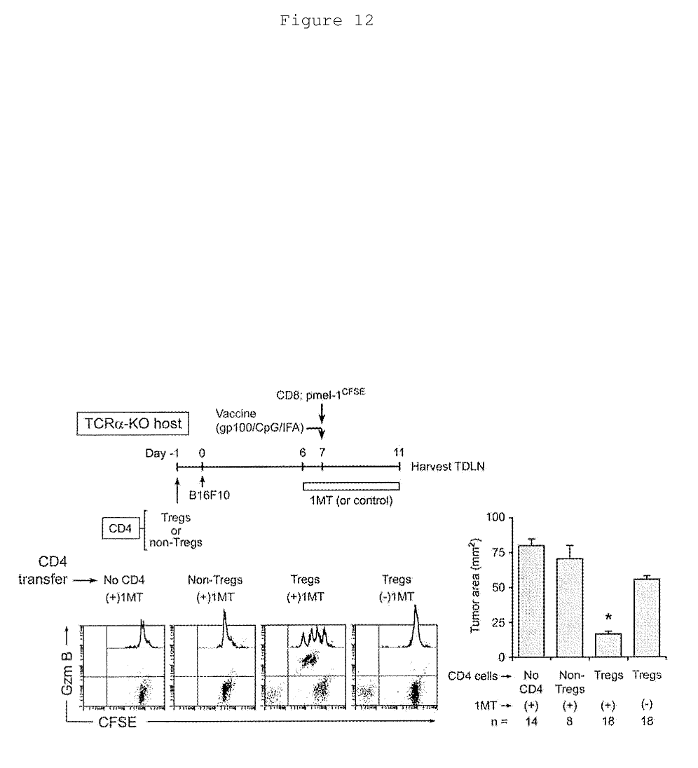

We next asked whether the beneficial effect of D1MT was mediated via its ability to restore Treg reprogramming. Pmel-1 cells were informative in this regard, because they require help from CD4⁺ cells for optimal anti-tumor efficacy. In FIG. 12B, TCRα-KO host mice were pre-loaded with different CD4⁺ populations: either sorted Foxp3^{GFP} Tregs, GFP^{NEG} non-Tregs, or no CD4⁺ cells. Mice were implanted with B16F10 tumors, and on day 7 received pmel-1 T cells and gp100 immunization, with or without D1MT administration. In the absence of any CD4⁺ cells (first dot-plot), pmel-1 cells showed little response to vaccination, despite D1MT administration. Transfer of conventional (non-Treg) CD4⁺ cells provided no detectable help for pmel-1 (second dot-plot), even with D1MT. Only the Treg fraction supported proliferation of pmel-1 cells, granzyme B upregulation, and anti-tumor effect (third dot-plot). The beneficial effects of Tregs was entirely lost if D1MT was omitted (last dot-plot), consistent with the observation that Tregs were unable to undergo normal reprogramming when IDO was active (FIG. 11). Thus, the beneficial effects of D1MT On anti-tumor vaccination appeared strictly dependent on Treg-derived helper activity.

### Example 18

### Helper Activity of Reprogrammed Tregs for Anti-Tumor Responses Requires CD40L

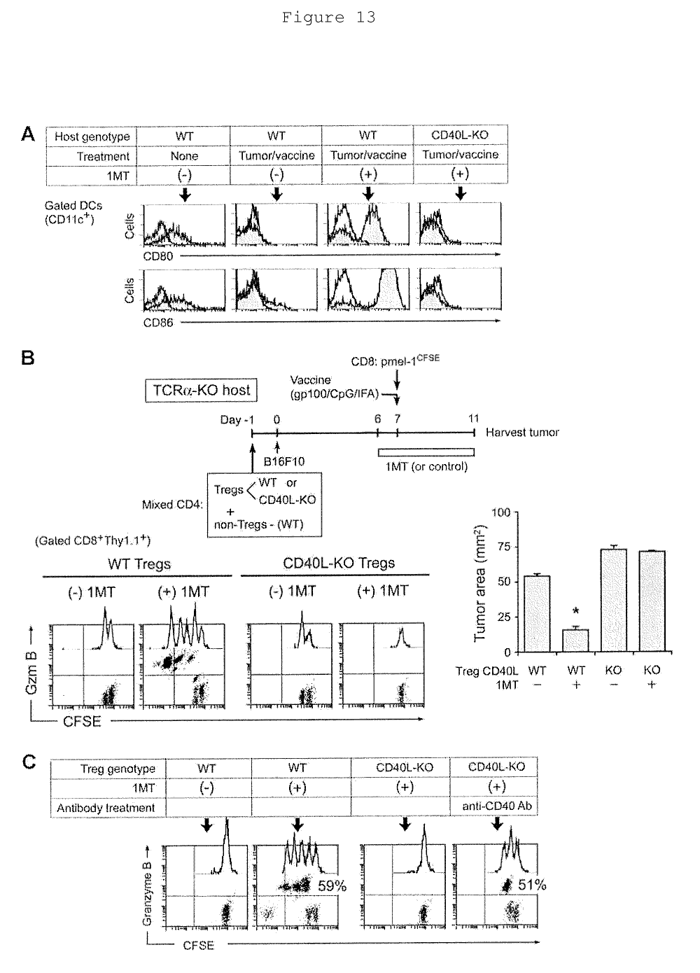

Tumors are known to suppress DC activation in tumor-draining LNs; and we knew from the studies in FIG. 9B that CD40L, was required in order for reprogrammed Tregs to activate DCs. Therefore, we asked whether CD40L was required for DC activation when tumor-bearing mice were treated with vaccine+D1MT. In resting control mice (without tumors), DCs expressed basal low levels of CD80 and CD86 (FIG. 13A, first set of histograms). In mice with established tumors, this expression was almost completely lost following vaccination without D1MT (second set of histograms). In contrast, if mice were treated with D1MT at the time of vaccination, then DC expression of CD80 and CD86 was upregulated at high levels (third set of histograms). The ability of D1MT to restore expression of CD80/CD86 was entirely lost in host mice lacking CD40L (fourth set of histograms). Thus, CD40L appeared to be an important downstream mediator of DC activation by D1MT+ vaccination.

We next asked whether the relevant site of CD40L expression was specifically on the reprogrammed Tregs. TCRα-KO hosts were pre-loaded with a defined mixture of sorted Tregs plus sorted non-Treg CD4⁺ cells (FIG. 13B). Half of the mice received Tregs from CD40L-KO donors, and half received Tregs from WT donors (CD40L, intact). All mice received the non-Treg fraction (GFP^{NEG}) from Foxp3^{GFP} mice, which have intact CD40L. Thus, following reconstitution, all host cells and all non-Treg CD4⁺ cells had intact CD40L, and the groups differed only in whether the Treg population could express CD40L. All mice then received pmel-1 and gp100 vaccination, with or without D1MT as shown. FIG. 13B shows that only those mice receiving CD40L-sufficient Tregs but not those mice receiving CD40L-KO Tregs—were able to support full CD8⁺ T cell responses in the presence of D1MT (extensive proliferation, granzyme B expression and anti-tumor activity). Thus, CD40L was required for CD4 helper activity in this model, and the relevant source of CD40L was specifically the Treg population. (In these studies, the CD4⁺CD25⁺ Treg preparation contained a small number of contaminating non-Tregs; however, this was immaterial because all mice already received a large excess of CD40L-competent non-Tregs, with no effect.)

Finally, to directly test the mechanistic role of CD40 ligation in mediating the helper activity of reprogrammed Tregs, FIG. 13C shows that the defect in helper activity of CD40L-KO Tregs could be substantially rescued by treating mice with cross-linking anti-CD40 antibody. The antibody was somewhat less effective than the native CD40L, but it restored proliferation and granzyme B expression, thus supporting a direct mechanistic contribution of the CD40 pathway.

### REFERENCES

Below are a list of references which support the disclosure above, all of which are incorporated by reference in their entireties.
Alexander et al., 2002; Diabetes, 51:356
Ahonen et al., 2004; J. Exp. Med., 199:775-784
Antony, et al. 2005; J. Immunol. 174:2591-2601
Baban et al. 2005; Int. Immunol, 17:909-919
Bennett et al., 1998; Nature, 393:478-480
Bennett, et al., 1997; J. Exp. Med., 186:65-70
Chen, 2005; Curr Opin Organ Transplant, 10:181-185
Descombes, et al., 1991; Cell, 67:569-579
Dranoff, 2002; Immunol Rev., 188:147-54
Eberl, et al., 2004; Nat. Immunol., 5:64-73
Falo, et al., 1995; Nat. Med., 1:649-653
Fehervari and Sakaguchi 2004; Int Immunol, 16:1769-1780
Fisson et al. 2003; J Exp Med, 198:737-746
Fontenot, et al., 2005; Immunity, 22:329-341
Friberg et al., 2002; nternational Journal of Cancer, 101:151-155
Gilliet and Liu. 2002; J Exp Med, 195:695-704
Grohmann et al., 2001 J. Immunol., 167:708
Grohmann et al., 2001; J. Immunol., 166:277
Grohmann et al., 2003; Trends in Immunology, 24:242-248
Grohmann et al., 2003; J. Exp. Med., 198:153-160
Grohmann et al. 2002; Nature Immunology, 3:985-1109
Hemmi et al., 2000; Nature, 408:740-745
Hsieh et al. 2004; Immunity, 21:267-277

Hwu et al., 2000; J. Immunol., 164:3596-3599
Khattri et al. 2003; Nat Immunol, 4:337-342
Kraj et al., 2001; J. Exp. Med., 194:407-416
Kronenberg et al. 2005; Nature, 435:598-604
Lee et al., 2003; Laboratory Investigation, 83:1457-1466
Liu, 2005 Annu Rev Immunol 23:275-306
Martin et al., 2002; Blood, 100:383-390
Mellor et al., 2002; J. Immunol., 168:3771
Mellor et al., 2003; J. Immunol., 171:1652-1655
Mellor et al., 2005; J. Immunol., 175:5601-5605
Mellor, et al. 2008; Nat. Rev. Immunol., 8:74-80
Miconnet, et al., 2002; J. Immunol., 176:6503-6511
Mild et al., 2001; Transplantation Proceedings, 33:129-130
Moseman et al., 2004; J. Immunol., 173:4433-4442
Muller et al., 2005; Nat. Med, 11:312-319
Munn et. al., 2004; J. Clin Invest., 114:280-290
Munn et al., 2005; Immunity, 22:1-10
Munn et al., 1998; Science, 281:1191;
Munn et al., 1999; J. Exp. Med., 189:1363
Munn et al., 2002; Science, 297:1867-1870
Munn et al., 2005; Immunity, 22:633-642
Nishikawa et al. 2005; J Exp Med, 201:681-686
Oranona et al., 2004; Nat. Immunol., 5:1134-1142

Overwijk et al., 2003; J. Exp. Med., 198:569-580
Pasare and Medzhitov, 2003; Science, 299:1033-1036
Peterson et al., 1993; Med Chem Res, 4:473-482
Ridgway, 2003; Cancer Invest., 21:873-86
Sakaguchi, 2005; Nat Immunol, 6:345-352
Schoenberger et al., 1998; Nature, 393:480-483
Shurin et al., 1997; Cell Immunol., 179:174-184
Sono et al., 1996; (Them Rev, 96:2841
Sono et al., 1989; Biochemistry, 28:5392-9
Southan et al, 1996; Med. Chem. Res., 343-352
Stone and Darlington, 2002; Nat Rev Drug Discov, 1:609-620
Swanson, et al. 2004; Am J Respir Cell Mol Biol, 30:311-8
Taylor et al., 1991; FASEB Journal, 5:2516-2522
Taylor et al., 2001; J. Exp. Med., 193:1311-1317
Thornton, et al., 2004; Eur. J. Immunol., 34:366-376
Uyttenhove et al., 2003; Nature Medicine, 9:1269-1274
Vottero et al., 2006; Biotechnol J., 1:282-288
Wei et al., 2005; Cancer Res, 65:5020-5026
Zou, Nat. Rev. Immunol. 206; 6:295-307

1. A method of reprogramming a regulatory T cell (Treg) to acquire a pro-inflammatory T-helper-like phenotype comprising exposing said Treg to a sufficient quantity of a vaccine and an inhibitor of the IDO pathway such that said reprogramming occurs.

2. (canceled)

3. The method of claim 1, wherein said vaccine comprises an antigenic protein or a nucleic acid encoding the same.

4. The method of claim 1, wherein said vaccine is a viral vector vaccine.

5. The method of claim 4, wherein said viral vaccine is a lentiviral vaccine.

6. The method of claim 5, wherein said lentiviral vaccine encodes a tumor antigen.

7-9. (canceled)

10. The method of claim 1, wherein said vaccine is administered prior to, concurrently with, or after said IDO inhibitor.

11. The method of claim 1, wherein said IDO inhibitor is formulated for oral delivery.

12. The method of claim 11, wherein said IDO inhibitor is formulated as a powder, capsule, tablet or liquid.

13. The method of claim 1, wherein said IDO inhibitor is selected from the group consisting of 1-methyl-tryptophan, 1-methyl-D-tryptophan, and 1-methyl-L-tryptophan.

14. (canceled)

15. (canceled)

16. A method of reprogramming a regulatory T cell (Treg) to acquire a pro-inflammatory T-helper-like phenotype in a subject comprising exposing said subject to a sufficient quantity of B7 ligand and IDO inhibitor such that said reprogramming occurs.

17. The method of claim 16 wherein said B7 ligand is CD28-Ig.

18. The method of claim 16 wherein the IDO inhibitor is selected from the group consisting of 1-methyl-tryptophan, 1-methyl-D-tryptophan, and 1-methyl-L-tryptophan.

19-23. (canceled)

24. A method to increase the immune response elicited by a vaccine, the method consisting in administering to a patient a vaccine plus 1-methyl-D-tryptophan.

25. The method of claim 24, wherein said vaccine is an isolated protein in combination with adjuvants.

26. The method of claim 25, wherein said adjuvant is CpG oligonucleotides.

27. The method of claim 24, wherein the vaccine is a lentivirus vaccine.

28. A method to induce conversion of FoxP3+ regulatory T cells into pro-inflamatory T-helper-like cells in an individual said method consisting in administration of a vaccine plus an inhibitor of the IDO pathway to said individual.

29. The method of claim 28, where said inhibitor of the IDO pathway is 1-methyl-D-tryptophan.

30. The method of claim 28, wherein said vaccine is an isolated protein in combination with adjuvants.

31. The method of claim 30, wherein said adjuvant is CpG oligonucleotides.

32-45. (canceled)


SEQUENCE LISTING (3 sequences)

SEQ ID NO: 1 - DNA, 20 bp, Unknown
  CpG-1826 phosphorothioate oligo
tccatgacgttcctgagctt

SEQ ID NO: 2 - protein, 9 aa, Homo sapiens
KVPRNQDWL

SEQ ID NO: 3 - protein, 8 aa, Unknown
  Ovalbumin antigen
SIINFEKL